SEQUENCE LISTING (3 sequences)

SEQ ID NO: 1 - DNA, 3382 bp, Homo sapiens
  CDS (245)...(3103)
tttttatgggaatcgcagcttggaagagacagarcaattccagaawtaaattgraattgaagatttaaccaatgttgttttaaaatattctaacttcaaagaatgatgccagaacttwaaaagggrctgcgcagagtagcaggggccctggagggcgcggcctgaatcctgattgcccttctgctgagaggacacacgcagctgaagatgaatttgggaaaagtagccgcttgctactttaactatggaagagcagggccacagtgagatggaaataatcccatcagagtctcacccccacattcaattactgaaaagcaatcgggaacttctggtcactcacatccgcaatactcagtgtctggtggacaacttgctgaagaatgactacttctcggccgaagatgcggagattgtgtgtgcctgccccacccagcctgacaaggtccgcaaaattctggacctggtacagagcaagggcgaggaggtgtccgagttcttcctctacttgctccagcaactcgcagatgcctacgtggacctcaggccttggctgctggagatcggcttctccccttccctgctcactcagagcaaagtcgtggtcaacactgacccagtgagcaggtatacccagcagctgcgacaccatctgggccgtgactccaagttcgtgctgtgctatgcccagaaggaggagctgctgctggaggagatctacatggacaccatcatggagctggttggcttcagcaatgagagcctgggcagcctgaacagcctggcctgcctcctggaccacaccaccggcatcctcaatgagcagggtgagaccatcttcatcctgggtgatgctggggtgggcaagtccatgctgctacagcggctgcagagcctctgggccacgggccggctagacgcaggggtcaaattcttcttccactttcgctgccgcatgttcagctgcttcaaggaaagtgacaggctgtgtctgcaggacctgctcttcaagcactactgctacccagagcgggaccccgaggaggtgtttgccttcctgctgcgcttcccccacgtggccctcttcaccttcgatggcctggacgagctgcactcggacttggacctgagccgcgtgcctgacagctcctgcccctgggagcctgcccaccccctggtcttgctggccaacctgctcagtgggaagctgctcaagggggctagcaagctgctcacagcccgcacaggcatcgaggtcccgcgccagttcctgcggaagaaggtgcttctccggggcttctcccccagccacctgcgcgcctatgccaggaggatgttccccgagcgggccctgcaggaccgcctgctgagccagctggaggccaaccccaacctctgcagcctgtgctctgtgcccctcttctgctggatcatcttccggtgcttccagcacttccgtgctgcctttgaaggctcaccacagctgcccgactgcacgatgaccctgacagatgtcttcctcctggtcactgaggtccatctgaacaggatgcagcccagcagcctggtgcagcggaacacacgcagcccagtggagaccctccacgccggccgggacactctgtgctcgctggggcaggtggcccaccggggcatggagaagagcctctttgtcttcacccaggaggaggtgcaggcctccgggctgcaggagagagacatgcagctgggcttcctgcgggctttgccggagctgggccccgggggtgaccagcagtcctatgagtttttccacctcaccctccaggccttctttacagccttcttcctcgtgctggacgacagggtgggcactcaggagctgctcaggttcttccaggagtggatgccccctgcgggggcagcgaccacgtcctgctatcctcccttcctcccgttccagtgcctgcagggcagtggtccggcgcgggaagacctcttcaagaacaaggatcacttccagttcaccaacctcttcctgtgcgggctgttgtccaaagccaaacagaaactcctgcggcatctggtgcccgcggcagccctgaggagaaagcgcaaggccctgtgggcacacctgttttccagcctgcggggctacctgaagagcctgccccgcgttcaggtcgaaagcttcaaccaggtgcaggccatgcccacgttcatctggatgctgcgctgcatctacgagacacagagccagaaggtggggcagctggcggccaggggcatctgcgccaactacctcaagctgacctactgcaacgcctgctcggccgactgcagcgccctctccttcgtcctgcatcacttccccaagcggctggccctagacctagacaacaacaatctcaacgactacggcgtgcgggagctgcagccctgcttcagccgcctcactgttctcagactcagcgtaaaccagatcactgacggtggggtaaaggtgctaagcgaagagctgaccaaatacaaaattgtgacctatttgggtttatacaacaaccagatcaccgatgtcggagccaggtacgtcaccaaaatcctggatgaatgcaaaggcctcacgcatcttaaactgggaaaaaacaaaataacaagtgaaggagggaagtatctcgccctggctgtgaagaacagcaaatcaatctctgaggttgggatgtggggcaatcaagttggggatgaaggagcaaaagccttcgcagaggctctgcggaaccaccccagcttgaccaccctgagtcttgcgtccaacggcatctccacagaaggaggaaagagccttgcgagggccctgcagcagaacacgtctctagaaatactgtggctgacccaaaatgaactcaacgatgaagtggcagagagtttggcagaaatgttgaaagtcaaccagacgttaaagcatttatggcttatccagaatcagatcacagctaaggggactgcccagctggcagatgcgttacagagcaacactggcataacagagatttgcctaaatggaaacctgataaaaccagaggaggccaaagtctatgaagatgagaagcggattatctgtttctgagaggatgctttcctgttcatggggtttttgccctggagcctcagcagcaaatgccactctgggcagtcttttgtgtcagtgtcttaaaggggcctgcgcaggcgggactatcaggagtccactgcctycatgatgcaagccagcttcctgtgcagaaggtctggtcggcaaactccctaagtacccgctacaattctgcagaaaaagaatgtgtcttgcgagctgttgtagttacagtaaatacactgtgaagagaaaaaaaaaacggacgcgtgg

SEQ ID NO: 2 - protein, 953 aa, Homo sapiens
MEEQGHSEMEIIPSESHPHIQLLKSNRELLVTHIRNTQCLVDNLLKNDYFSAEDAEIVCACPTQPDKVRKILDLVQSKGEEVSEFFLYLLQQLADAYVDLRPWLLEIGFSPSLLTQSKVVVNTDPVSRYTQQLRHHLGRDSKFVLCYAQKEELLLEEIYMDTIMELVGFSNESLGSLNSLACLLDHTTGILNEQGETIFILGDAGVGKSMLLQRLQSLWATGRLDAGVKFFFHFRCRMFSCFKESDRLCLQDLLFKHYCYPERDPEEVFAFLLRFPHVALFTFDGLDELHSDLDLSRVPDSSCPWEPAHPLVLLANLLSGKLLKGASKLLTARTGIEVPRQFLRKKVLLRGFSPSHLRAYARRMFPERALQDRLLSQLEANPNLCSLCSVPLFCWIIFRCFQHFRAAFEGSPQLPDCTMTLTDVFLLVTEVHLNRMQPSSLVQRNTRSPVETLHAGRDTLCSLGQVAHRGMEKSLFVFTQEEVQASGLQERDMQLGFLRALPELGPGGDQQSYEFFHLTLQAFFTAFFLVLDDRVGTQELLRFFQEWMPPAGAATTSCYPPFLPFQCLQGSGPAREDLFKNKDHFQFTNLFLCGLLSKAKQKLLRHLVPAAALRRKRKALWAHLFSSLRGYLKSLPRVQVESFNQVQAMPTFIWMLRCIYETQSQKVGQLAARGICANYLKLTYCNACSADCSALSFVLHHFPKRLALDLDNNNLNDYGVRELQPCFSRLTVLRLSVNQITDGGVKVLSEELTKYKIVTYLGLYNNQITDVGARYVTKILDECKGLTHLKLGKNKITSEGGKYLALAVKNSKSISEVGMWGNQVGDEGAKAFAEALRNHPSLTTLSLASNGISTEGGKSLARALQQNTSLEILWLTQNELNDEVAESLAEMLKVNQTLKHLWLIQNQITAKGTAQLADALQSNTGITEICLNGNLIKPEEAKVYEDEKRIICF

SEQ ID NO: 3 - DNA, 2859 bp, Homo sapiens
atggaagagcagggccacagtgagatggaaataatcccatcagagtctcacccccacattcaattactgaaaagcaatcgggaacttctggtcactcacatccgcaatactcagtgtctggtggacaacttgctgaagaatgactacttctcggccgaagatgcggagattgtgtgtgcctgccccacccagcctgacaaggtccgcaaaattctggacctggtacagagcaagggcgaggaggtgtccgagttcttcctctacttgctccagcaactcgcagatgcctacgtggacctcaggccttggctgctggagatcggcttctccccttccctgctcactcagagcaaagtcgtggtcaacactgacccagtgagcaggtatacccagcagctgcgacaccatctgggccgtgactccaagttcgtgctgtgctatgcccagaaggaggagctgctgctggaggagatctacatggacaccatcatggagctggttggcttcagcaatgagagcctgggcagcctgaacagcctggcctgcctcctggaccacaccaccggcatcctcaatgagcagggtgagaccatcttcatcctgggtgatgctggggtgggcaagtccatgctgctacagcggctgcagagcctctgggccacgggccggctagacgcaggggtcaaattcttcttccactttcgctgccgcatgttcagctgcttcaaggaaagtgacaggctgtgtctgcaggacctgctcttcaagcactactgctacccagagcgggaccccgaggaggtgtttgccttcctgctgcgcttcccccacgtggccctcttcaccttcgatggcctggacgagctgcactcggacttggacctgagccgcgtgcctgacagctcctgcccctgggagcctgcccaccccctggtcttgctggccaacctgctcagtgggaagctgctcaagggggctagcaagctgctcacagcccgcacaggcatcgaggtcccgcgccagttcctgcggaagaaggtgcttctccggggcttctcccccagccacctgcgcgcctatgccaggaggatgttccccgagcgggccctgcaggaccgcctgctgagccagctggaggccaaccccaacctctgcagcctgtgctctgtgcccctcttctgctggatcatcttccggtgcttccagcacttccgtgctgcctttgaaggctcaccacagctgcccgactgcacgatgaccctgacagatgtcttcctcctggtcactgaggtccatctgaacaggatgcagcccagcagcctggtgcagcggaacacacgcagcccagtggagaccctccacgccggccgggacactctgtgctcgctggggcaggtggcccaccggggcatggagaagagcctctttgtcttcacccaggaggaggtgcaggcctccgggctgcaggagagagacatgcagctgggcttcctgcgggctttgccggagctgggccccgggggtgaccagcagtcctatgagtttttccacctcaccctccaggccttctttacagccttcttcctcgtgctggacgacagggtgggcactcaggagctgctcaggttcttccaggagtggatgccccctgcgggggcagcgaccacgtcctgctatcctcccttcctcccgttccagtgcctgcagggcagtggtccggcgcgggaagacctcttcaagaacaaggatcacttccagttcaccaacctcttcctgtgcgggctgttgtccaaagccaaacagaaactcctgcggcatctggtgcccgcggcagccctgaggagaaagcgcaaggccctgtgggcacacctgttttccagcctgcggggctacctgaagagcctgccccgcgttcaggtcgaaagcttcaaccaggtgcaggccatgcccacgttcatctggatgctgcgctgcatctacgagacacagagccagaaggtggggcagctggcggccaggggcatctgcgccaactacctcaagctgacctactgcaacgcctgctcggccgactgcagcgccctctccttcgtcctgcatcacttccccaagcggctggccctagacctagacaacaacaatctcaacgactacggcgtgcgggagctgcagccctgcttcagccgcctcactgttctcagactcagcgtaaaccagatcactgacggtggggtaaaggtgctaagcgaagagctgaccaaatacaaaattgtgacctatttgggtttatacaacaaccagatcaccgatgtcggagccaggtacgtcaccaaaatcctggatgaatgcaaaggcctcacgcatcttaaactgggaaaaaacaaaataacaagtgaaggagggaagtatctcgccctggctgtgaagaacagcaaatcaatctctgaggttgggatgtggggcaatcaagttggggatgaaggagcaaaagccttcgcagaggctctgcggaaccaccccagcttgaccaccctgagtcttgcgtccaacggcatctccacagaaggaggaaagagccttgcgagggccctgcagcagaacacgtctctagaaatactgtggctgacccaaaatgaactcaacgatgaagtggcagagagtttggcagaaatgttgaaagtcaaccagacgttaaagcatttatggcttatccagaatcagatcacagctaaggggactgcccagctggcagatgcgttacagagcaacactggcataacagagatttgcctaaatggaaacctgataaaaccagaggaggccaaagtctatgaagatgagaagcggattatctgtttc

(19) United States
(12) Patent Application Publication
(10) Pub. No.: US 2005/0287612 A1
(43) Pub. Date: Dec. 29, 2005

(54) CARD-4 molecules and uses thereof

(76) Inventors: John Bertin, Winchester, MA (US); Dana Philpott, Paris (FR); Philippe Sansonetti, Paris (FR); Stephen Girardin, Paris (FR)

Correspondence Address: FISH & RICHARDSON PC, P.O. BOX 1022, MINNEAPOLIS, MN, 55440-1022, US

(21) Appl. No.: 10/966,846

(22) Filed: Oct. 15, 2004

Related U.S. Application Data
(63) Continuation of application No. 10/706,857, filed on Nov. 12, 2003, now abandoned.
Application No. 10/706,857 is a continuation of application No. 10/352,381, filed on Jan. 27, 2003, now abandoned.
Application No. 10/352,381 is a continuation of application No. 10/154,485, filed on May 22, 2002, now abandoned.
Application No. 10/154,485 is a continuation-in-part of application No. 10/027,881, filed on Dec. 20, 2001, now abandoned.
(60) Provisional application No. 60/258,724, filed on Dec. 29, 2000.

(51) Int. Cl. G01N 33/554; G01N 33/569

(52) U.S. Cl. 435/7.32

(57) ABSTRACT

The invention provides methods of using CARD4 molecules to screen for modulators of LPS-induced cell signaling pathways. Also included are CARD-4 deficient mice, methods of modulating LPS-induced cell signaling pathways, and methods of treating or preventing bacterial infections.

### RELATED APPLICATIONS

This application is a continuation-in-part and claims priority to U.S. application Ser. No. 10/027,881, filed on Dec. 20, 2001, which claims priority to U.S. provisional application No. 60/258,724, filed on Dec. 29, 2000, the contents of which are incorporated herein by reference.

### BACKGROUND OF THE INVENTION

Innate immune recognition of bacterial products is an ancient system of host defense that shares striking similarities in species as diverse as humans, fruit flies and plants (Kopp et al. (1999) Curr Opin Immunol 11:13-18). It is not surprising that the bacterial products recognized are invariant molecules, including structural components such as lipopolysaccharide (LPS) of Gram negative organisms and peptidoglycan (PG) from the cell walls of Gram-positive organisms (Kopp et al. (1999) Curr Opin Immunol 11:13-18). Collectively, these microbial products are termed pathogen-associated molecular patterns or PAMPs. A family of receptors termed Toll/Toll-like receptors (TLRs) is central to innate immunity in both Drosophila and humans. Plants detect invading pathogens through a class of membrane-bound and cytosolic molecules termed disease-resistance proteins or R proteins, which also exhibit a striking resemblance to TLRs. The N protein of the tobacco plant, for example, although being cytosolic, possesses a C-terminal TIR domain common to TLRs as well as an N-terminal leucine rich repeat (LRR) domain similar to that of the extracellular portion of the TLRs (Medzhitov et al. (1998) Curr Opin Immunol 10:12-15). These R proteins mediate the hypersensitive response in plants resulting in metabolic alterations and localized cell death at the site of pathogen entry.

In contrast to what is known about TLRs in mediating PAMP responsiveness in cells of the myeloid lineage, the role played by TLRs in pathogen recognition in epithelial cells remains poorly defined. Epithelial cells may express TLRs, however, their function in innate immune detection is unclear as these cells are largely unresponsive to extracellular LPS and to non-pathogenic bacteria (Cario et al. (2000) J Immunol 164:966; Philpott et al. (2000) J Immunol 165:903). In contrast, intracellular LPS, either microinjected or delivered into the cytosol by invasive Shigella flexneri, is a potent inducer of the inflammatory response as assessed by the activation of NF-κB and one of its target genes, IL-8 (Philpott et al. (2000) J Immunol 165:903). The mechanism by which intracellular LPS activates these responses, however, has not yet been determined.

Nuclear factor-κB (NF-κB) is a transcription factor expressed in many cell types and which activates homologous or heterologous genes that have κB sites in their promoters. Molecules that regulate NF-κB activation play a critical role in both apoptosis and inflammation. Quiescent NF-κB resides in the cytoplasm as a heterodimer of proteins referred toas p50 and p65 and is complexed with the regulatory protein IκB. NF-κB binding to IκB causes NF-κB to remain in the cytoplasm. At least two dozen stimuli that activate NF-κB are known (New England Journal of Medicine 336:1066, 1997) and they include cytokines, protein kinase C activators, oxidants, viruses, and immune system stimuli. NF-κB activating stimuli activate specific IκB kinases that phosphorylate IκB leading to its degradation. Once liberated from IκB, NF-κB translocates to the nucleus and activates genes with κB sites in their promoters. The proinflammatory cytokines TNF-α and IL-1 induce NF-κB activation by binding their cell-surface receptors and activating the NF-κB-inducing kinase, NIK, and NF-κB. NIK phosphorylates the IκB kinases a and which phosphorylate IκB, leading to its degradation.

NF-κB and the NF-κB pathway has been implicated in mediating chronic inflammation in inflammatory diseases such as asthma, ulcerative colitis, rheumatoid arthritis (Epstein, New England Journal of Medicine 336:1066, 1997) and inhibiting NT-κB or NF-κB pathways may be an effective way of treating these diseases. NT-κB and the NF-κB pathway has also been implicated in atherosclerosis (Navab et al., Amerinan Journal of Cardiology 76:18C, 1995), especially in mediating fatty streak formation, and inhibiting NF-κB or NF-κB pathways may be an effective therapy for atherosclerosis. Among the genes activated by NF-κB are cIAP-1, cIAP-2, TRAF1, and TRAF2, all of which have been shown to protect cells from TNF-α induced cell death (Wang et al., Science 281:1680-83, 1998). CLAP, a protein which includes a CARD, activates the Apaf-1-caspase-9 pathway and activates NF-κB by acting upstream of NIK and IκB kinase (Srinivasula et al., supra).

### SUMMARY OF THE INVENTION

The present invention is based, at least in part, on the discovery that CARD-4 is involved in innate immune responses mediated through the activation of NF-κB and JNK and that CARD-4 participates in immune responses to bacterial infections.

A cDNA of CARD-4 described herein (SEQ ID NO:1) has a 2859 nucleotide open reading frame (nucleotides 245-3103 of SEQ ID NO:1; SEQ ID NO:3) which encodes a 953 amino acid protein (SEQ ID NO:2). CARD-4 possesses a CARD domain (amino acids 15-114 of SEQ ID NO:2). Human CARD-4 also has a nucleotide binding domain which extends from about amino acid 198 to about amino acid 397 of SEQ ID NO:2: a Walker Box “A”, which extends from about amino acid 202 to about amino acid 209 of SEQ ID NO:2; a Walker Box “B”, which extends from about amino acid 280 to about amino acid 284 of SEQ ID NO:2; a kinase 1a (P-loop) subdomain, which extends from about amino acid 127 to about amino acid 212 of SEQ ID NO:2; a kinase 2 subdomain, which extends from about amino acid 273 to about amino acid 288 of SEQ ID NO:2; a kinase 3a subdomain, which extends from about amino acid 327 to about amino acid 338 of SEQ ID NO:2: and ten Leucine-rich repeats which extend from about amino acid 674 to about amino acid 950 of SEQ ID NO:2. The first Leucine-rich repeat extends from about amino acid 674 to about amino acid 701 of SEQ ID NO:2. The second Leucine-rich repeat extends from about amino acid 702 to about amino acid 727 of SEQ ID NO:2. The third Leucine-rich repeat extends from about amino acid 728 to about amino acid 754 of SEQ ID NO:2. The fourth Leucine-rich repeat extends from about amino acid 755 to about amino acid 782 of SEQ ID NO:2. The fifth Leucine-rich repeat extends from about amino acid 783 to about amino acid 810 of SEQ ID NO:2. The sixth Leucine-rich repeat extends from about amino acid 811 to about amino acid 838 of SEQ ID NO:2. The seventh Leucine-rich repeat extends from about amino acid 839 to about amino acid 866 of SEQ ID NO:2. The eighth Leucine-rich repeat extends from about amino acid 867 to about amino acid 894 of SEQ ID NO:2. The ninth Leucine-rich repeat extends from about amino acid 895 to about amino acid 922 of SEQ ID NO:2. The tenth leucine-rich repeat extends from about amino acid 923 to about amino acid 950 of SEQ ID NO:2.

In addition to the CARD-4 sequences described herein, CARD-4 amino acid or nucleotide sequences described in U.S. Pat. Nos. 6,340,576 or 6,369,196. the entire contents of which are incorporated by reference, can also be used in the practice of the invention. For example, any of the CARD-4 sequences or fragments thereof (e.g., a functional domain such as a CARD, NBS, or LRR domain) described in U.S. Pat. Nos. 6,340,576 or 6,369,196 can be used in the methods described herein.

CARD-4 activates the NF-kB pathway and enhances caspase-9 activity. In addition, CARD-4 associates with CARD-4, CARD-3, caspase 9, and BCLX. Upon activation. CARD-4 likely binds a nucleotide, thus allowing CARD-4 to bind and activate a CARD-containing caspase via a CARD-CARD interaction, leading to the activation of inflammatory and/or apoptotic signaling pathways in the cell. CARD-4 is described in detail in U.S. Pat. Nos. 6,340,576 or 6,369,196.

The invention encompasses methods of diagnosing and treating individuals having a bacterial infection or a disorder of bacterial origin. Bacterial pathogens include but are not limited to, bacteria of Mycobacterium species, Helicobacter species (e.g., Helicobacter pylori), Salmonella species (e.g., Salmonella typhimurium), Shigella species (e.g., Shigella flexneri), E. coli, Rickettsia species, Listeria species, Legionella species (e.g., Legionella pneumoniae), Pseudomonas species, Vibrio species, and Borellia species (e.g., Borellia burgdorferi). Disorders of bacterial origin include Bacterial infections of the upper respiratory tract (e.g., nasopharyngitis, sinusitis, purulent and acute otitis media, peritonsillar abscess), chronic obstructive pulmonary disease (e.g., emphysema and bronchitis), bacterial infections of the central nervous system (e.g., bacterial meningitis, subdural empyema, and septic thrombophlebitis) sepsis, inflammatory bowel disease (e.g.. bacillary dysentery, Crohn's disease, ulcerative colitis, ischemic colitis, diverticulitis or diveniculotis, and appendicitis), and sepsis and septic shock.

The activation of NF-kB and JNK by CARD-4 in response to bacterial LPS is expected to lead to the production of several important mediators of innate immunity, such as cytokines and chemokines. Accordingly, immune responsiveness can be modulated (increased or decreased) by modulating (increasing or decreasing) the expression or activity of CARD-4 in a cell.

Bacterial LPS is a cell-wall component of gram-negative bacteria that has the ability to induce a dramatic systemic reaction known as septic shock. This syndrome is the result of overwhelming secretion of cytokines, particularly of TNF-α, often as a result of an uncontrolled systemic bacterial infection. It is expected that septic shock can be prevented or treated by interfering with LPS-induced activation of CARD-4. For example, inhibiting CARD-4 induced activation of NF-kB is expected to reduce or inhibit symptoms associated with septic shock.

The invention encompasses methods of treatment that modulate the CARD-4 signaling pathways described herein that are activated by LPS and bacterial infection. Also included in the invention are methods of screening for modulators (activators or inhibitors) of these CARD-4 pathways.

The invention also encompasses methods of diagnosing and treating patients who are suffering from a disorder associated with an abnormal level or rate (undesirably high or undesirably low) of apoptotic cell death, abnormal activity of the Fas/APO-1 receptor complex, abnormal activity of the TNF receptor complex, or abnormal activity of a caspase by administering a compound that modulates the expression of CARD-4 (at the DNA, mRNA or protein level, e.g., by altering mRNA splicing) or by altering the activity of CARD-4. Examples of such compounds include small molecules, antisense nucleic acid molecules, ribozymes, and polypeptides.

Certain disorders are associated with an increased number of surviving cells. which are produced and continue to survive or proliferate when apoptosis is inhibited or occurs at an undesirably low rate. Compounds that modulate the expression or activity of CARD-4 can be used to treat or diagnose such disorders. These disorders include cancer (particularly follicular lymphomas, chronic myelogenous leukemia, melanoma, colon cancer, lung carcinoma, carcinomas associated with mutations in p53, and hormone-dependent tumors such as breast cancer, prostate cancer, and ovarian cancer). Such compounds can also be used to treat viral infections (such as those caused by herpesviruses, poxviruses, and adenoviruses). Failure to remove autoimmune cells that arise during development or that develop as a result of somatic mutation during an immune response can result in autoimmune disease. Thus, autoimmune disorders can be caused by an undesirably low levels of apoptosis. Accordingly, modulators of CARD-4 activity or expression can be used to treat autoimmune disorders (e.g., systemic lupus erythematosis, immune-mediated glomerulonephritis, and arthritis).

Many diseases are associated with an undesirably high rate of apoptosis. Modulators of CARD-4 expression or activity can be used to treat or diagnose such disorders. For example, populations of cells are often depleted in the event of viral infection, with perhaps the most dramatic example being the cell depletion caused by the human immunodeficiency virus (HIV). Surprisingly, most T cells that die during HIV infections do not appear to be infected with HIV. Although a number of explanations have been proposed, recent evidence suggests that stimulation of the CD4 receptor results in the enhanced susceptibility of uninfected T cells to undergo apoptosis. A wide variety of neurological diseases are characterized by the gradual loss of specific sets of neurons. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

CARD-4 polypeptides, nucleic acids and modulators of CARD-4 expression or activity can be used to treat immune disorders. Such immune disorders include, but are not limited to, chronic inflammatory diseases and disorders, such as inflammatory bowel disease (e.g., Crohn's disease and ulcerative colitis); arthritis, including reactive arthritis (e.g., Lyme disease) and rheumatoid arthritis; insulin-dependent diabetes; organ-specific autoimmunity, including multiple sclerosis, Hashimoto's thyroiditis and Grave's disease; contact dermatitis; psoriasis; graft rejection; graft versus host disease; sarcoidosis; atopic conditions, such as asthma and allergy, including allergic rhinitis; gastrointestinal allergies, including food allergies; eosinophilia; conjunctivitis; glomerular nephritis; certain pathogen susceptibilities, such as helminthic infections (e.g., leishmaniasis), certain viral infections, including HIV, and bacterial infections, including tuberculosis and lepromatous leprosy; inflammatory disorders of the respiratory tract, including bronchitis and chronic obstructive pulmonary disease.

In addition to the aforementioned disorders, CARD-4 polypeptides, nucleic acids, and modulators of CARD-4 expression or activity can be used to treat disorders of cell signaling and disorders of tissues in which CARD-4 is expressed.

A CARD-4 nucleic acid includes a nucleic acid molecule which is at least 45% (or 55%, 65%, 75%. 85%, 95%, or 98%) identical to the nucleotide sequence shown in SEQ ID NO: 1, SEQ ID NO:3, or a complement thereof.

A CARD-4 nucleic acid includes a nucleic acid molecule which includes a fragment of at least 150 (300, 325, 350, 375, 400, 425, 450, 500, 550, 600, 650, 700, 800, 900, 1000, 1300, 1600 or 1931) nucleotides of the nucleotide sequence shown in SEQ ID NO: 1. SEQ ID NO:3, or a complement thereof.

A CARD-4 nucleic acid includes a nucleic acid molecule which includes a nucleotide sequence encoding a protein having an amino acid sequence that is at least 45% (or 55%, 65%, 75%, 85%, 95%, or 98%) identical to the amino acid sequence of SEQ ID NO:2.

A CARD-4 nucleic acid includes a nucleic acid molecule w hich encodes a fragment of a polypeptide having the amino acid sequence of SEQ ID NO:2, the fragment including at least 15 (25, 30, 50, 100, 150, 300, 400 or 540, 600, 700, 800, 900) contiguous amino acids of SEQ ID NO:2.

A CARD-4 nucleic acid includes a nucleic acid molecule which encodes a naturally occurring allelic variant of a polypeptide comprising the amino acid sequence of SEQ ID NO:2. In general, an allelic variant of a gene will be readily identifiable as mapping to the same chromosomal location as said gene. For example, the chromosomal location of the human CARD-4 gene is on chromosome 7 close to the SHGC-31928 genetic marker. Allelic variants of human CARD-4 will be readily identifiable as mapping to the human CARD-4 locus on chromosome 7 near genetic marker SHGC-31928.

A CARD-4 protein includes: an isolated CARD-4 protein having an amino acid sequence that is at least about 65%, preferably 75%, 85%, 95%, or 98% identical to the amino acid sequence of SEQ ID NO:2; an isolated CARD-4 protein having an amino acid sequence that is at least about 85%, 95%, or 98% identical to the CARD domain of SEQ ID NO:2 (e.g., about amino acid residues 15 to 114 of SEQ ID NO:2); an isolated CARD-4 protein having an amino acid sequence that is at least about 85%, 95%, or 98% identical to the nucleotide binding domain of SEQ ID NO:2 (e.g., about amino acid residues 198 to 397 of SEQ ID NO:2; an isolated CARD-4 protein having an amino acid sequence that is at least about 85%, 95%, or 98% identical to the kinase 1a (P-loop) subdomain SEQ ID NO:2 (e.g., about amino acid 127 to about amino acid 212 of SEQ ID NO:2); an isolated CARD-4 protein having an amino acid sequence that is at least about 85%, 95%, or 98% identical to the kinase 2 subdomain of SEQ ID NO:2 (e.g., about amino acid 273 to about amino acid 288 of SEQ ID NO:2); an isolated CARD-4 protein having an amino acid sequence that is at least about 85%, 95%, or 98% identical to a kinase 3a subdomain of SEQ DD NO:2 (e.g., about amino acid residues 327 to 338 of SEQ ID NO:2); an isolated CARD-4 protein having an amino acid sequence that is at least about 85%, 95%, or 98% identical to the Leucine-rich repeats of SEQ ID NO:2 (e.g., about amino acid residues 674 to 701 of SEQ ID NO:2, from amino acid 702 to amino acid 727 of SEQ ID NO:2; from amino acid 728 to amino acid 754 SEQ ID NO:2; from amino acid 755 to amino acid 782 of SEQ ID NO:2; from amino acid 783 to amino acid 810 of SEQ ID NO:2; from amino acid 81I1 to amino acid 838 of SEQ ID NO:2; from amino acid 839 to amino acid 866 of SEQ ID NO:2; from amino acid 867 to amino acid 894 of SEQ ID NO:2; from amino acid 895 to amino acid 922 of SEQ ID NO:2, and from amino acid 923 to amino acid 950 of SEQ ID NO:2).

CARD-4 nucleic acid molecules can be used to specifically detect CARD-4 nucleic acid molecules, relative to nucleic acid molecules encoding other members of the CARD superfamily. For example, in one embodiment, a CARD-4 nucleic acid molecule hybridizes under stringent conditions to a nucleic acid molecule comprising the nucleotide sequence of SEQ ID NO:1, SEQ ID NO:3, or a complement thereof. In another embodiment, the CARD-4 nucleic acid molecule is at least 300 (350, 400, 450, 500, 550, 600, 650, 700, 800, 900, 1000, 1300, 1600, 1900, 2100, 2400, 2700, 3000, or 3382) nucleotides in length and hybridizes under-stringent conditions to a nucleic acid molecule comprising the nucleotide sequence shown in SEQ ID NO:1, SEQ ID NO:3, or a complement thereof. In another embodiment, an isolated CARD-4 nucleic acid molecule comprises nucleotides 287 to 586 of SEQ ID NO:1, encoding the CARD domain of CARD-4, or a complement thereof. In yet another embodiment, the invention provides an isolated nucleic acid molecule which is antisense to the coding strand of a CARD-4 nucleic acid.

A vector, e.g., a recombinant expression vector, comprising a CARD-4 nucleic acid can be used in the methods of the invention. In another embodiment the invention provides a host cell containing such a vector. The invention also provides a method for producing CARD-4 protein by culturing, in a suitable medium, a host cell of the invention containing a recombinant expression vector such that a CARD-4 protein is produced.

Preferred CARD-4 proteins and polypeptides possess at least one biological activity possessed by naturally occurring human CARD-4, e.g., (1) the ability to form protein:protein interactions with proteins in the apoptotic signalling pathway; (2) the ability to form CARD-CARD interactions with proteins in the apoptotic signaling pathway; (3) the ability to bind a CARD-4 ligand (e.g., CARD-4, CARD-3, caspase 9, and/or BCLX); (4) the ability to bind to an intracellular target; (5) the ability to enhance caspase 9 activity; and (6) the ability to activate the NF-kB pathway. Other activities include: (1) modulation of cellular proliferation; (2) modulation of cellular differentiation; (3) modulation of cellular death; and (4) modulation of inflammation and/or an innate immune response.

A CARD-4 protein, or biologically active portions thereof, can be operatively linked to a non-CARD-4 polypeptide (e.g., heterologous amino acid sequences) to form CARD-4 fusion proteins, respectively. The invention further features antibodies that specifically bind CARD-4 proteins, such as monoclonal or polyclonal antibodies. In addition, the CARD-4 proteins or biologically active portions thereof can be incorporated into pharmaceutical compositions, which optionally include pharmaceutically acceptable carriers.

The invention provides a method for detecting the presence of CARD-4 activity or expression in a biological sample by contacting the biological sample faith an agent capable of detecting an indicator of CARD-4 activity such that the presence of CARD-4 activity is detected in the biological sample.

In another aspect, the invention pro,-ides a method for modulating CARD-4 activity comprising contacting a cell with an agent that modulates (inhibits or stimulates) CARD-4 activity or expression such that CARD-4 activity or expression in the cell is modulated. In one embodiment, the agent is an antibody that specifically binds to CARD-4 protein. In another embodiment, the agent modulates expression of CARD-4 by modulating transcription of a CARD-4 gene, splicing of a CARD-4 mRNA, or translation of a CARD-4 mRNA. In yet another embodiment, the agent is a nucleic acid molecule having a nucleotide sequence that is antisense to the coding strand of the CARD-4 mRNA or the CARD-4 gene.

In one embodiment, the methods of the present invention are used to treat a subject having a disorder characterized by aberrant CARD-4 protein or nucleic acid expression or activity or related to CARD-4 expression or activity by administering an agent which is a CARD-4 modulator to the subject. In one embodiment, the CARD-4 modulator is a CARD-4 protein. In another embodiment the CARD-4 modulator is a CARD-4 nucleic acid molecule. In other embodiments, the CARD-4 modulator is a peptide, peptidomimetic, or other small molecule.

The present invention also provides a diagnostic assay for identifying the presence or absence of a genetic lesion or mutation characterized by at least one of: (i) aberrant modification or mutation of a gene encoding a CARD-4 protein; (ii) mis-regulation of a gene encoding a CARD-4 protein; (iii) aberrant RNA splicing; and (iv) aberrant post-translational modification of a CARD-4 protein, wherein a wild-type form of the gene encodes 3 protein with a CARD-4 activity.

In another aspect, the invention provides a method for identifying a compound that binds to-or modulates the activity of a CARD-4 protein. In general, such methods entail measuring a biological activity of a CARD-4 protein in the presence and absence of a test compound and identifying those compounds which alter the activity of the CARD-4 protein.

The invention also features methods for identifying a compound which modulates the expression of CARD-4 by measuring the expression of CARD-4 in the presence and absence of a compound.

The invention also features methods for identifying compounds (e.g., small molecules, peptide, etc.) that interfere with CARD-4 mediated activation of NK-κB and/or JNK. Suitable assays for NF-κB and JNK activation are described below. In addition, activation can be assessed by measuring the level of phospho-c-Jun in cell nuclei using, for example, the assay described below. In the case of NK-κB activation, it can be desirable to measure TNF-α mediated activation in order to determine if the activation of NF-κB is relatively CARD-4 specific.

In addition, the invention features methods for identifying compounds that interfere with the response of cells, e.g., epithelial cells to LPS. Such compounds may be identified by identifying compounds which interfere with CARD-4 oligomerization (or oligomerization of the CARD domain of CARD-4) in the presence or absence of LPS. Such assays can be carried out in vivo or in vitro. The invention also features methods for treating toxic shock or inflammation associated with intracellular pathogens by blocking CARD-4 activation or activity. For example, it can be desirable to administer a compound that interferes with nucleotide binding to the NBS domain of CARD-4. Inhibitors of CARD-4 activity can include polypeptides that act as dominant negative mutants. For example, a variant CARD-4 lacking the CARD domain.

The invention features a method for identifying a candidate compound that modulates lipopolysaccharide (LPS)-mediated activation of NT-κB, the method comprising providing a cell that harbors LPS and expresses a polypeptide comprising a caspase recruitment domain (CARD), nucleotide binding site (NBS); or leucine rich repeat (LRR) domain of CARD-4; exposing the cell to a test compound; and measuring NF-kB activation in the cell: wherein altered NF-kB activation in the presence of the test compound compared to NT-kB activation in the absence of the test compound indicates that the test compound is a candidate compound that modulates LPS-mediated activation of NF-kB. In some embodiments, the cell is infected with Shigella flexneri, Salmonella typhimurium, or Helicobacter pylori.

The invention also features a method for identifying a candidate compound that modulates LPS-mediated activation of JNK kinase activity, the method comprising: providing a cell that harbors LPS and expresses a polypeptide comprising a CAR, NBS, or LRR domain of CARD-4; exposing the cell to a test compound; and measurings JNK kinase activity in the cell; wherein altered JNK kinase activity in the presence of the test compound compared to JNK kinase activity in the absence of the test compound indicates that the test compound is a candidate compound that modulates LPS-mediated activation of JNK kinase activity. In some embodiments, the cell is infected with Shigella flexneri, Salmonella typhimurium, or Helicobacter pylori.

The invention also features a method for identifying a candidate compound that modulates an LPS-induced immune-response, the method comprising: providing a cell that expresses a polypeptide comprising a CARD, NBS, or LRR domain of CARD-4; introducing LPS into the cell; exposing the cell to a test compound; and measuring oligomerization of the polypeptide in the cell; wherein altered oligomerization of the polypeptide in the presence of the test compound compared to oligomerization of the polypeptide in the absence of the test compound indicates that the test compound is a candidate compound for modulating an LPS-induced immune response. In some embodiments, the cell is infected with Shigella flexneri, Salmonella typhimurium, or Helicobacter pylori.

The polypeptide used in the screening methods described herein can optionally be a recombinant polypeptide. The methods can optionally include an additional step of introducing into the cell a heterologous nucleic acid encoding the polypeptide. The polypeptide can correspond in sequence to a naturally-occurring or a non-naturally-occurring CARD-4 sequence.

The invention also features a method of modulating LPS-induced activation of NF-kB or JNK, the method comprising: providing a cell that harbors intracellular LPS; and contacting the cell with a compound that modulates expression or activity of CARD-4 in an amount sufficient to modulate LPS-induced activation of NF-kB or JNK in the cell.

The invention also features a method of modulating an LPS-induced immune response in an individual, the method comprising: selecting an individual comprising cells harboring intracellular LPS; and administering to the individual a compound that modulates expression or activity of CARD-4 in an amount sufficient to modulate an LPS-induced immune response in the individual. In some embodiments, the individual is diagnosed as having a bacterial infection, e.g., a Shigella flexneri infection, Salmonella typhimurium infection, or Helicobacter pylori infection.

The invention also features a method of treating or preventing a bacterial infection, the method comprising: selecting an individual having or at risk of having a bacterial infection; administering to the individual a compound that modulates expression or activity of CARD-4 in an amount sufficient to treat or prevent the bacterial infection.

The invention also features a mouse whose genome comprises a disruption in an endogenous CARD-4 gene, wherein said disruption results in decreased expression or a lack of expression of said endogenous CARD-4 gene, thereby causing a decreased ability of the mouse to clear a Salmonella typhimurium or Helicobacter pylori infection.

Other features and advantages of the invention will be apparent from the following detailed description and claims.

### BRIEF DESCRIPTION OF THE DRAWINGS

FIGS. 1A-1F depict a cDNA sequence (SEQ ID NO:1) and predicted amino acid sequence (SEQ D NO:2) of human CARD-4. The open reading frame of CARD-4 extends from nucleotide 245 to nucleotide 3103 of SEQ ID NO:1 (SEQ ID NO:3).

FIG. 2A depicts the results of an experiment demonstrating that S. flexneri infection leads to the phosphorylation of cJun.

FIG. 2B depicts the results of an experiment demonstrating that microinjection of LPS activates c-Jun phosphorylation.

FIG. 3A depicts the results of an experiment demonstrating that a dominant-negative form of TRAF2 does not inhibit the induction of NF-κB by invasive S. flexneri.

FIG. 3B depicts the results of an experiment demonstrating that a dominant-negative form of TRAF6 does not inhibit the induction of NF-κB by invasive S. flexneri.

FIG. 3C depicts the results of an experiment demonstrating that S. flexneri induces NF-κB in HEK293 deficient in IL-1 specific signaling components.

FIG. 3D depicts the results of an experiment demonstrating that the dominant-negative molecule MyD88 (DN-MyD88) does not inhibit NF-κB activation by invasive S. flexneri.

FIG. 4A depicts the domain structure of CARD-4 compared with plant disease resistance proteins tobacco N protein and Arabidopsis RPS2 protein.

FIG. 4B depicts the result of an experiment demonstrating that CARD-4 oligomerizes following S. flexneri infection.

FIG. 5A depicts the results of an experiment demonstrating that ΔCARD CARD-4 inhibits NF-κB induction by S. flexneri but not TNFα.

FIG. 5B depicts the results of an experiment demonstrating that isolated CARD-4 LRR domain inhibits NF-κB induction by S. flexneri but not TNFα.

FIG. 5C depicts the result of an experiment depicting the effect of the overexpression of full-length and truncated forms of CARD-4 on JNK activation.

FIG. 6 depicts the bacterial load of wild type mice and CARD-4 deficient mice following infection with Salmonella typhimurium.

FIG. 7 depicts the bacterial load of wild type mice and CARD-4 deficient mice following infection with Helicobacter pylori.

### DETAILED DESCRIPTION OF THE INVENTION

The present invention is based, in part, on the discovery that CARD-4 is involved in innate immune responses mediated through the activation of NF-κB and JNK and that CARD-4 participates in immune responses to bacterial infections.

CARD-4 Mediates NF-κB and JNK Activation in Response to Shigella Flexneri and Intracellular LPS

Experiments were conducted to determine whether cellular responses to intracellular LPS include the activation of c-Jun N-terminal kinase (JNK), a kinase important in the stress response to numerous stimuli. The target of JNK, c-Jun, is phosphorylated by this kinase on serine 63 and 73, an event that increases its ability to activate transcription. c-Jun is a component of the transcription factor AP-1, another important regulator in the inflammatory response (Foletta et al. (1998) J Leukoc Biol 63:139-52).

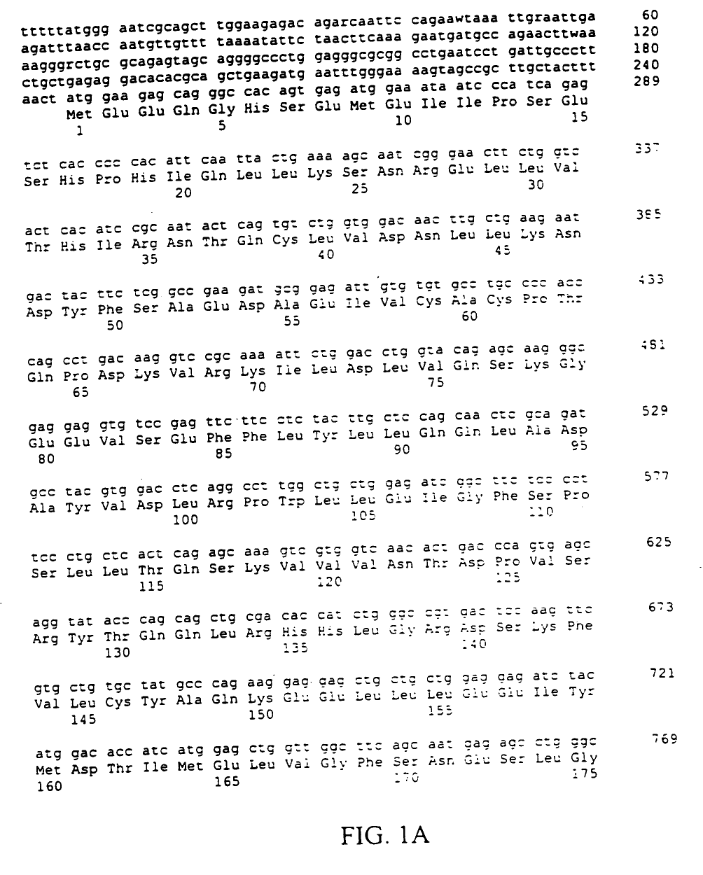

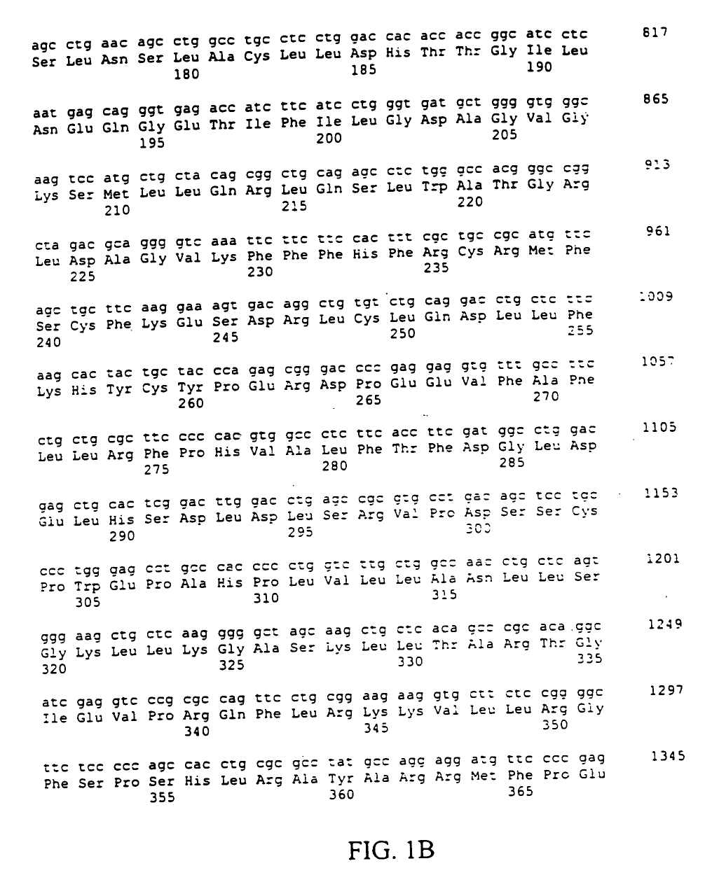

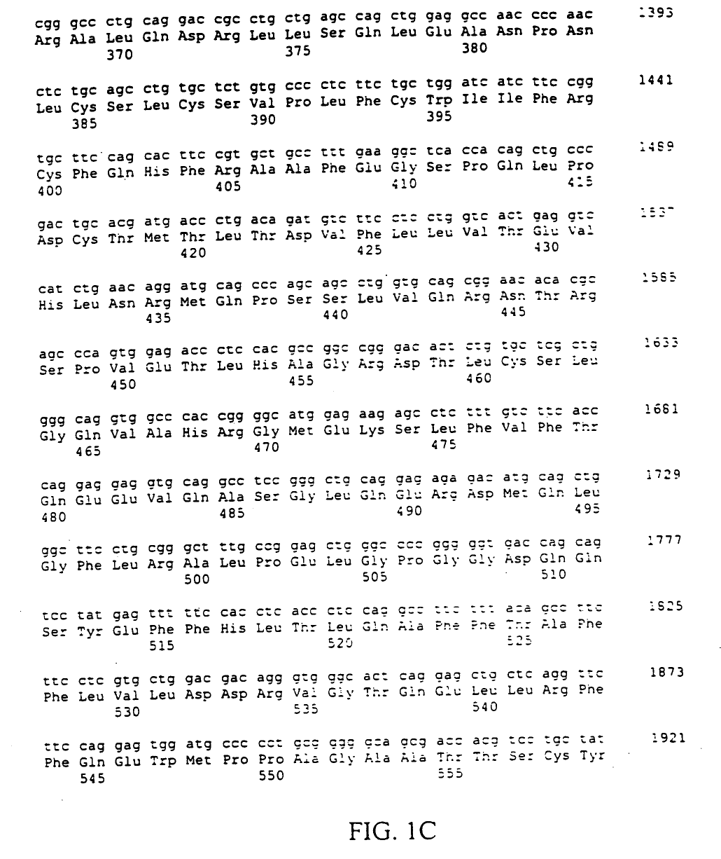

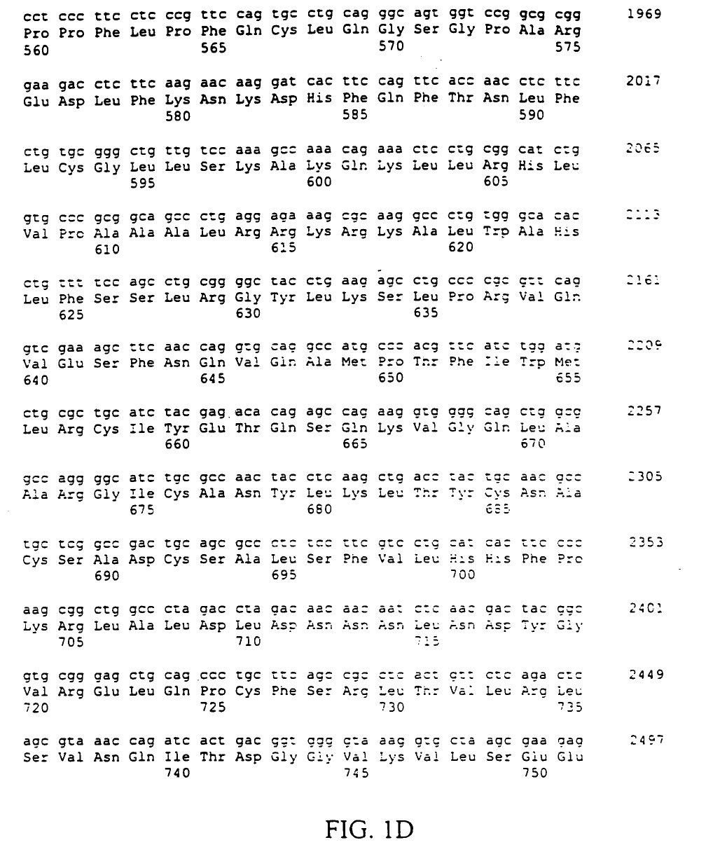

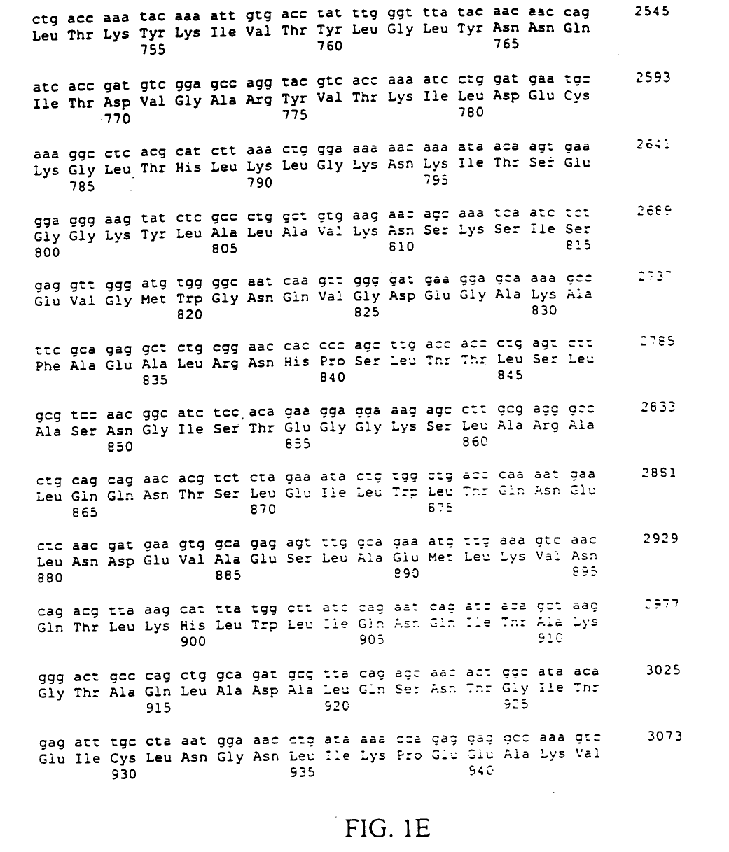

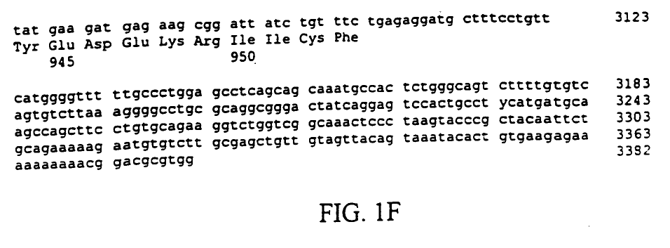

HeLa cells were infected with wild-type invasive S. flexneri strain M90T or the plasmid-cured, noninvasive strain BS176, for 2 hours as described previously (Philpott et al. (2000) J Immunol 165:903-14). Infected cells were then stained for both phospho-c-Jun using a monoclonal phospho-specific antibody to this protein (Lallemand et al. (1998) EMBO J 17:5615) and LPS to label the infecting bacteria using a rabbit polyclonal anti LPS antibody. Stained cells were viewed using conventional fluorescence microscopy. The immunofluorescent staining of infected cells with an antibody specific for c-Jun phosphorylated on serine 63 suggested that JNK was activated by wild-type S. flexneri leading to the nuclear accumulation of phospho-c-Jun (FIG. 1A). In contrast, the non-invasive strain did not induce c-Jun phosphorylation (FIG. 1A). As previously shown for NF-κB induction (Philpott et al. (2000) J Immunol 165:903), activation of JNK by S. flexneri could be mimicked by microinjection of LPS (FIG. 1B). Briefly, HeLa cells were microinjected with FITC-dextran to identify microinjected cells plus buffer alone or purified Escherichia coli LPS 0111:B4 as previously described (Philpott et al. (2000) J Immunol 165:903). Following a 1 hour incubation at 37° C. and 5% CO₂, cells were stained for phospho-c-Jun and examined by fluorescence microscopy.

The intracellular detection system present in epithelial cells for sensing LPS was investigated by examining the signal transduction pathway leading from intracellular LPS detection to NF-κB and JNK activation. Much is known about the signaling pathways induced by TNFα and IL-1 and both of these signals result in the activation of NF-κB and JNK (Song et al. (1997) Proc Natl Acad Sci 94:9792; O'Neill et al. (1998) J Leuko Biol 63:650). Moreover, the signaling pathway leading from extracellular LPS detection mediated through TLRs to NF-κB and JNK is identical to that of the IL-1 pathway (O'Neill et al. (1998) J Leuko Biol 63:650). Therefore, the possibility that the S. flexneri-induced signaling pathway shared signaling components common to the TNFα and IL-1 induced pathways was investigated. Dominant negative versions of the TLR/IL-1 specific signaling protein, TRAF6 (Cao et al. (1996) Nature 383:443), as well as the TNFα-specific protein, TRAF2 (Song et al. (1997) Proc Natl Acad Sci 94:9792), were over-expressed in epithelial cells and examined for their effect on S. flexneri-induced NF-κB activation using an NF-κB responsive luciferase gene reporter assay.

Briefly, HEK293 cells were transfected with vector alone or increasing amounts of DNA encoding for the dominant-negative forms of TRAF2 (DN-TRAF2) or TRAF6 (DN-TRAF6) along with a NF-κB luciferase reporter plasmid and a e-galactosidase plasmid to normalize transfection efficiencies. Following 30 hours, cells were infected with wild-type S. flexneri or treated with either TNFα (100 ng/ml) or IL-1 (10 ng/ml) for 4 hours and then assayed for luciferase activity as described previously (Philpott et al. (2000) J Immunol 165:903-14).

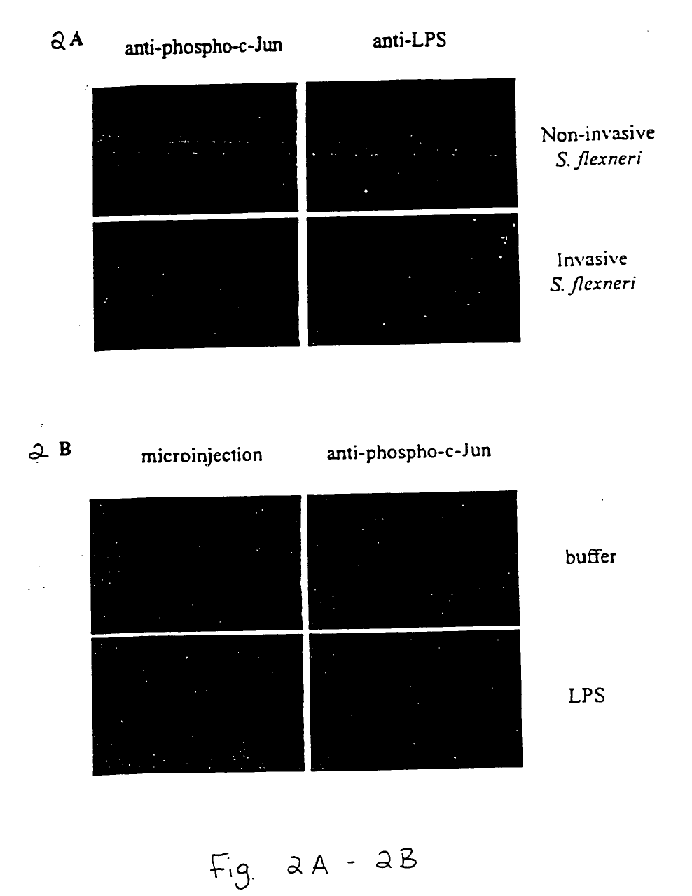

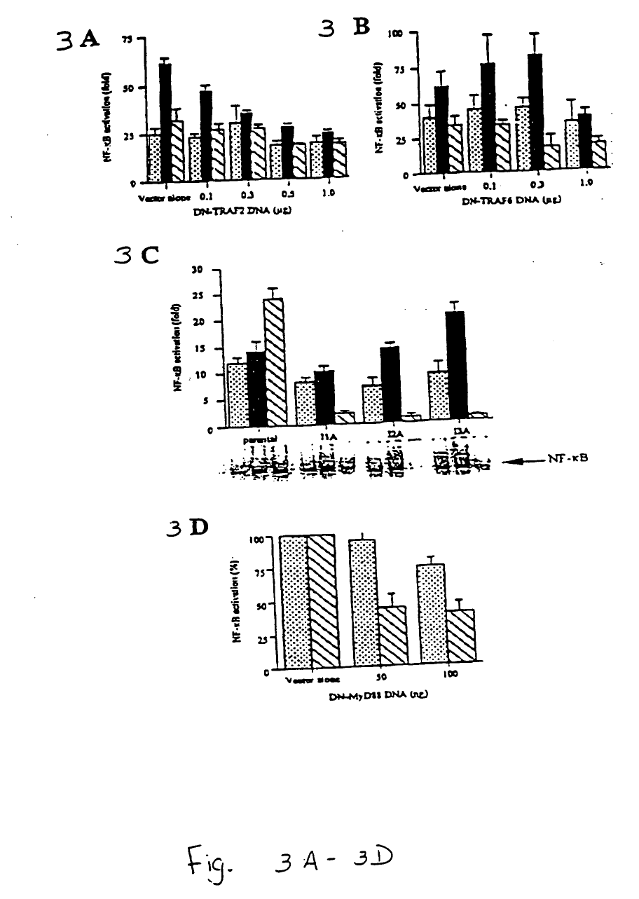

Neither dominant negative TRAF2 nor TRAF6 expression affected S. flexneri induction of the NF-κB luciferase reporter at concentrations that inhibited NT-κB induction by TNFα and IL-1, respectively (FIGS. 3A and 3B), and only marginal inhibition was observed with higher doses of dominant negative expressing plasmids.

These findings indicate that S. flexneri-induced NF-κB activation is TRAF2 and TRAF6 independent. It is clear, at least for TNFα that there may be functional redundancy among the TRAF family of recruitment factors. For example, mice deficient in TRAF2 are still capable of responding to TNFα (Yeh (1997) Immunity 7:715), and it is possible that redundancy also exists in TLR/IL-1 signaling pathways. Therefore, the possibility that factors in the TLR/IL-1 pathway upstream of TRAF6 are essential for NF-κB and JNK induction by S. flexneri infection was investigated. This was examined using three different HEK293 epithelial cell lines deficient in IL-1 signaling components upstream of TRAF6 (Li et al. (1999) Mol Cell Biol 19:4643). One of these cell lines, 11A, is deficient in interleukin-1 receptor associated kinase (IRAK), a factor that has been shown to be involved in LPS-induced activation of both NF-κB and JNK (Kanakaraj et al. (1998) J Exp Med 187:2037). The other two cell lines, 12A and 13A are deficient in unknown factors upstream of IRA (Li et al., (1999) Mol Cell Biol 19:4643) Briefly, NF-κB activation after S. flexneri infection, TNFα treatment or IL-1 treatment was compared in parental HEK293 cells and the three IL-1 signaling deficient cell lines. NF-κB activity was assessed following infection or cytokine treatment by the NF-iB reporter assay or EMSA (Philpon et al. (2000) J Immunol 165:903-14). S. flexneri infection, but not IL-1 treatment, activated both NF-iB (FIG. 3C) and JNK in 293 epithelial cells deficient in IRAK (11A cells) as well as in the two other IL-1 signalling deficient cell lines.

IRAK2, a homologue of IRAK, has been implicated in IL-1 signaling (Muzio et al. (1997) Science 278:1612) although its role in TLR mediated responsiveness to LPS has not been determined.

To examine whether IRAK might mediate NF-κB induction by S. flexneri in IRAK-deficient cells, dominant-negative IRAK2 was overexpressed in these cells (11A cells) and then infected with S. flexneri or treated with IL-1. Dominant-negative abolished the small induction of NF-iB activation seen following IL-1 treatment of IRAK-deficient cells, whereas this molecule had no effect on S. flexneri-induced activation. Taken together these findings indicate that S. flexneri-induced activation of NF-κB and JNK is both IRAK and IRAK2 independent.

MyD88 is an important adaptor protein involved in TLR/IL-1 signaling to NF-κB and JNK (O'Neill et al. (1998) J Leuko Biol 63:650). Indeed. MyD88 is critical for LPS responsiveness since mice deficient in this factor. e.g., the TLR4 mutant C3H/HeJ mice (Poltorak et al. (1998) 282:2085), are highly resistant to LPS (Kawai et al. (1999) Immunity 11:115). The role of MyD88 in NF-κB induction by invasive S. flexneri was, therefore, investigated. Briefly, increasing amounts of DNA encoding for dominant-negative MyD88 were transfected into HEK293 cells along with the NT-κB and β-galactosidase reporter plasmids and the cells were assayed for luciferase activity 4 hours later. NF-iB reporter assays were performed in duplicate at least three times. This study revealed that dominant-negative MyD88 inhibited IL-1-induced activation of an NF-iB luciferase reporter gene whereas S. flexneri-induced NT-κB activation was only marginally affected (FIG. 3D). Together, these findings indicate that S. flexneri-induced activation of NF-κB and JNK occurs via a pathway that is distinct from the TLR/IL-1 signaling pathway. Therefore, a distinct LPS detection and signal transduction system other than the TLRs likely exists in mammalian cells and this alternate system responds to intracellular LPS.

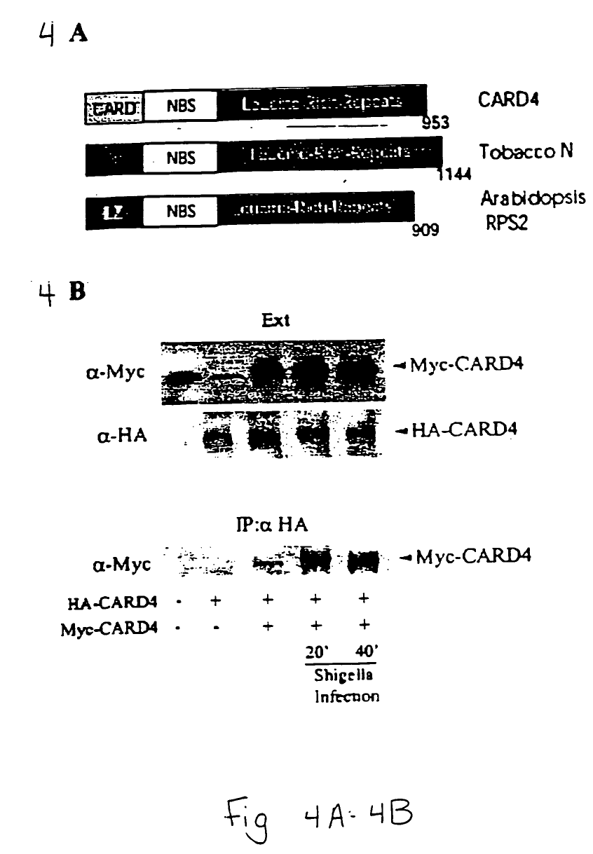

The LRR domain of CARD-4 contains significant amino acid similarity to that of several plant disease-resistance proteins, including tobacco N protein and Arabidopsis RPS2 protein, which also contains an NBS domain (FIG. 4A). Because of its similarity to plant R proteins and its ability to activate NF-κB, it was decided to investigate the role of CARD-4 in the detection of intracellular LPS in epithelial cells infected with S. flexneri. CARD-4 has structural and functional similarities to Apaf-1. For example, both proteins possess a CARD domain and an NBS domain. Moreover, both activate downstream regulators through self-oligomerization. A series of studies was carried out to determine whether S. flexneri infection could induce the activation of CARD-4 by enhancing its self-oligomerization. A full-length myc-tagged CARD-4 was co-expressed with a full-length hemagolutinin (HA) tagged CARD-4 in order to perform co-immunoprecipitation experiments following S. flexneri infection. Briefly, HeLa cells were transfected with empty vector or expression vectors encoding either HA-CARD-4 or Myc-CARD-4 for 24 hours and were left either uninfected or S. flexneri-infected for different times. Cells were collected and protein extracts (Ext) were subjected to Western blotting with rabbit polyclonal antibodies to Myc or HA (Santa Cruz Biotechnology) to identify the expression levels of the overexpressed proteins. Another fraction of the protein extracts was used for immunoprecipitation using a polyclonal anti-HA antibody. Oligomerized CARD-4 was revealed in the immunoprecipitates by Western blotting using antibodies to the Myc-tagged CARD-4. Enhanced self-association of CARD-4 was observed as early as 20 minutes post-infection with invasive S. flexneri (FIG. 4B). These studies provide biochemical evidence for the role of CARD-4 in intracellular pathogen recognition by epithelial cells.

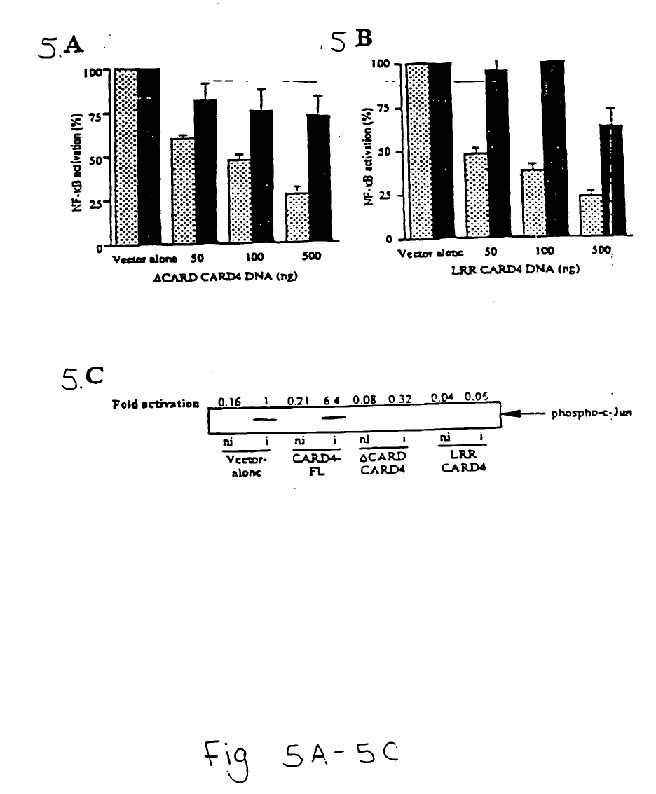

Experiments were conducted to determine whether the activation of CARD-4 induced by S. flexneri is the critical link between intracellular LPS detection and the activation of downstream signaling events including NF-κB and JNK activation. Since the CARD domain is necessary for NF-κB activation in CARD-4 overexpression studies, it is possible that a CARD-4 variant lacking this domain may act as a dominant-negative inhibitor of NF-κB and JNK induction by S. flexneri. Thus a series of experiments was conducted using CARD-4 deletion mutants. Briefly, a plasmid encoding either a CARD-4 variant lacking its CARD domain (ΔCARD CARD-4) or a CARD-4 variant lacking its LRR domain (ΔLRR CARD-4) was transfected into HEK293 cells along with NF-κB and β-galactosidase reporter plasmids. Luciferase activity was assayed 4 hours after infection with S. flexneri or TNFα treatment, as described above. Overexpression of ΔCARD CARD-4 molecule acted in a dose-dependent manner to inhibit S. flexneri-induced NF-κB activation (FIG. 5A).

Induction of the NF-κB reporter construct by TNFα was much less affected by overexpression of ΔCARD CARD-4, testifying to the relative specificity of this response. Furthermore, overexpression of the ΔCARD CARD-4 molecule blocked JNK induction by S. flexneri as assessed by an in vitro kinase assay (FIG. 5C). The dominant-negative effect induced by ΔCARD CARD-4 overexpression after S. flexneri infection is likely to be due to either an interference in the propagation of the signal following oligomerization of CARD-4 through the NBS domain or titration of the LPS-induced signaling pathway upstream of CARD-4 through the LRR domain.

An experiment in which the LRR domain of CARD-4 was overexpressed alone was conducted. Briefly, HeLa cells were transfected either with empty vector or with expression vectors encoding for CARD-4 full length (CARD-4-FL). ΔCARD CARD-4 or the LRR of CARD-4 for 40 hours. The cells were then infected with invasive (i) or non-invasive (ni) S. flexneri. After about 20 minutes the cells were collected and protein extracts analyzed using a JNK kinase assay. Fold activation of JNK normalized was to the S. flexneri-induced activation in vector only cells. This study revealed that the LRR domain when expressed alone inhibited both NF-κB and JNK activation by S. flexneri (FIGS. 5B and 5C). This result is consistent with the view that LRR domain overexpression interferes with upstream signaling pathways initiated by infection. This study also revealed that activation of MC in cells infected with S. flexneri was enhanced following overexpression of the full-length molecule (FIG. 5C) implying that low endogenous levels of this protein may restrict activation. Overexpression of the LRR domain of CARD-4 also inhibited signal transduction induced by intracellular LPS. In cells overexpressing the LRR domain, fewer cells exhibited activated NF-κB and phospho-c-Jun in the nuclei following LPS microinjection compared to cells expressing the vector alone (Table 1).

For the experiments summarized in Table 1, HeLa cells were transfected with either vector alone or the LRR domain of CARD-4 and microinjected with LPS (Philpott et al. (2000) J Immunol 165:903) 24 hours post-transfection and stained for phospho-c-Jun (as described above) or the p65 subunit of NF-iB (Philpott et al. (2000) J Immunol 165:903). At least 50 cells were counted for each condition. Taken together, these results implicate CARD-4 as a component of an intracellular LPS detection system capable of inducing innate immune responses mediated through the activation of NF-κB and JNK.

TABLE 1
Microinjection of LPS into transfected cells-examination
for induction of NF-κB and phospho-c-Jun.

| | | % of microinjected cells |
|---|---|---|
| Immunostaining | Transfection | activated |
| NF-κB | Vector | 96% |
| | LRR | 63% |
| Phospho-c-Jun | Vector | 71% |
| | LRR | 33% |

The results described herein indicate that a discriminatory system has evolved in epithelial cells based on the inside-versus-outside presentation of a pathogen-associated molecular pattern. Epithelial cells are refractory to extracellular LPS, yet the same molecule presented inside the cell is capable of initiating an inflammatory response. Thus, these cells have evolved a method of detecting intracellular PAMPs, similar to that in plants, and the findings presented here suggest that CARD-4 plays a significant role in this detection system.

CARD-4 is a member of a new family of proteins that possess a C-terminal LRR and an NBS. The “signaling module” domain, however, appears to be variable. This study on CARD-4 suggests the possibility that this family of proteins represents human homologues of plant disease-resistance proteins. These cytosolic proteins may be involved in mediating defensive responses to distinct intracellular pathogens or pathogen products. As it has been shown for the TLRs, different CARD-4-like proteins may exist that are involved in the recognition of distinct bacterial products.

Role CARD-4 in Bacterial Infections

To examine the role of CARD-4 in infectious processes in vivo, wild-type and CARD-4-deficient mice were infected with either Salmonella typhimurium or Helicobacter pylori and bacterial counts were assessed in various tissues following infection.

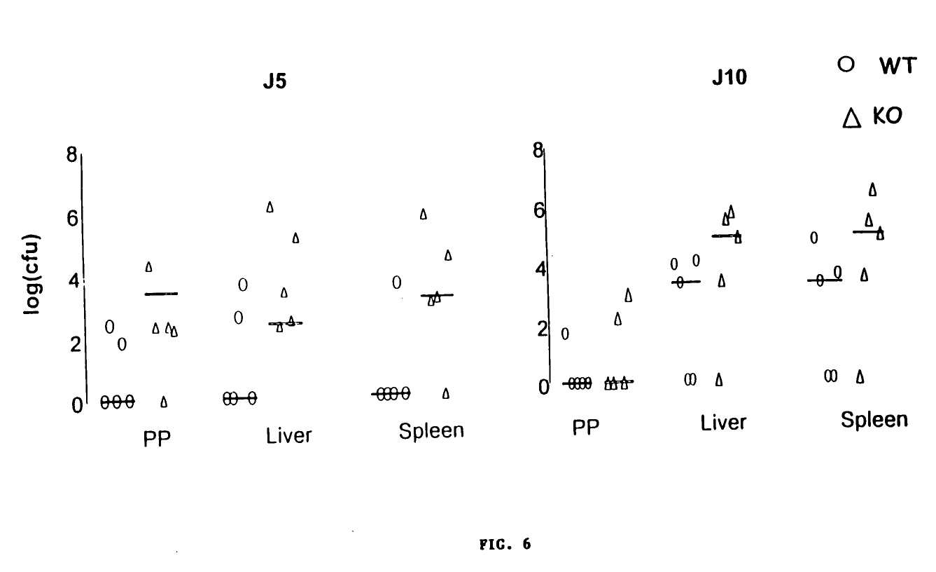

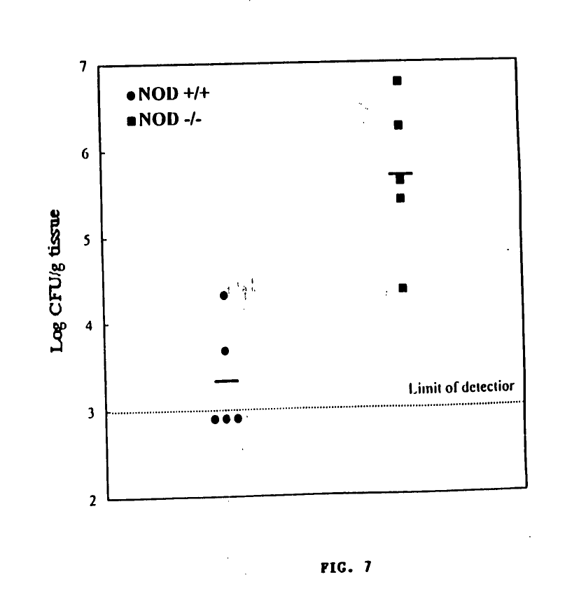

For the S. typhimurium infections, six week old wild-type or CARD-4-deficient mice were infected by the gastric route with 5×10⁷ colony-forming units (CFUs) of virulent S. typhimurium, strain C52. Peyer's patches, spleen and liver were collected after 5 or 10 days of infection. The tissues were ground, diluted and then plated in order to assess the number of CFUs in each organ. We observed that the bacterial load was higher in each organ in the CARD-4-deficient mice compared to the wild-type mice in each tissue at both day 5 and day 10, except in the Peyer's patches at day 10. These differences were highly significant, often more than 2 logs difference between CARD-4-deficient and wild-type mice (FIG. 6). These results suggest that CARD-4 plays an important role in bacterial clearance during S. typhimurium infection in mice.

For H. pylori infections, CARD-4-deficient and wild-type mice were infected by the gastric route with 1×10⁶ CFU of H. pylori strain SS 1, which is a human pathogenic strain adapted for mice infection. Stomachs were collected following 1 month of infection and the number of CFUs per gram of stomach tissue was assessed by plating serial diluted samples. On average, a 2 log difference was observed between the CARD-4-deficient mice compared to the wild-type mice (FIG. 7). These data further suggest a role for CARD-4 in the control of bacterial proliferation in the stomach and/or clearance of the infecting organism.

CARD-4 Proteins and Nucleic Acids

A nucleotide sequence encoding a human CARD-4 protein is shown in FIGS. 1A-1F (SEQ ID NO:1; SEQ DD NO:3 includes the open reading frame only ). A predicted amino acid sequence of CARD-4 protein is also shown in FIGS. 1A-1F (SEQ ID NO:2) The human CARD-4 cDNA of FIGS. 1A-1F has a molecular weight of approximately 108 kDa (excluding post-translational modifications).

A plasmid containing a cDNA encoding human CARD-4 (pC4L1) was deposited with the American Type Culture Collection (ATCC), Manasass, Va. on Jul. 7, 1998, and assigned Accession Number 203035. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

CARD-4 has a CARD domain (amino acids 15-114 of SEQ ID NO:2). CARD-4 also has a nucleotide binding domain which extends from about amino acid 198 to about amino acid 397 of SEQ ID NO:2; a predicted Walker Box “A”, which extends from about amino acid 202 to about amino acid 209 of SEQ ID NO:2, a predicted Walker Box “B”, which extends from about amino acid 280 to about amino acid 284 of SEQ ID NO:2; a predicted kinase 1a (P-loop) domain, which extends from about amino acid 197 to about amino acid 212 of SEQ ID NO:2; a predicted kinase 2 domain, which extends from about amino acid 273 to about amino acid 288 of SEQ ID NO:2; a predicted kinase 3a subdomain, which extends from about amino acid 327 to about amino acid 338 of SEQ ID NO:2; ten predicted Leucine-rich repeats which extend from about amino acid 674 to about amino acid 950 of SEQ ID NO:2. The first Leucine-rich repeat extends from about amino acid 674 to about amino acid 701 of SEQ ID NO:2. The second Leucine-rich repeat extends from about amino acid 702 to about amino acid 727 of SEQ ID NO:2. The third Leucine-rich repeat extends from about amino acid 728 to about amino acid 754 of SEQ ID NO:2. The fourth Leucine-rich repeat extends from about amino acid 755 to about amino acid 782 of SEQ ID NO:2. The fifth Leucine-rich repeat extends from about amino acid 783 to about amino acid 810 of SEQ ID NO:2. The sixth Leucine-rich repeat extends from about amino acid 811 to about amino acid 838 of SEQ ID NO:2. The seventh Leucine-rich repeat extends from about amino acid 839 to about amino acid 866 of SEQ ID NO:2. The eighth Leucine-rich repeat extends from about amino acid 867 to about amino acid 894 of SEQ ID NO:2. The ninth Leucine-rich repeat extends from about amino acid 895 to about amino acid 922 of SEQ ID NO:2. The tenth leucine-rich repeat extends from about amino acid 923 to about amino acid 950 of SEQ ID NO:2.

CARD-4 is a member of a family of molecules having certain conserved structural and functional features. The term “family” when referring to the protein and nucleic acid molecules of the invention is intended to mean two or more proteins or nucleic acid molecules having a common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

In one embodiment, a CARD-4 protein includes a CARD domain having at least about 65%, preferably at least about 75%, and more preferably about 85%, 95%, or 98% amino acid sequence identity to the CARD domain of SEQ ID NO:2.

Preferred CARD-4 polypeptides useful in the methods of the present invention have an amino acid sequence sufficiently identical to the CARD domain amino acid sequence of SEQ ID NO:2.

The CARD-4 polypeptide has an amino acid sequence sufficiently identical to the nucleotide binding domain of SEQ ID NO:2, an amino acid sequence sufficiently identical to the Walker Box “A” of SEQ ID NO:2 or Walker Box “B” of SEQ ID NO:2. an amino acid sequence sufficiently identical to the kinase 1a subdomain of SEQ ID NO:2, an amino acid sequence sufficiently identical to the kinase 2 subdomain of SEQ ID NO:2, or an amino acid sequence sufficiently identical to the kinase 3a subdomain of SEQ ID NO:2, or an amino acid sequence sufficiently identical to the Leucine-rich repeats of SEQ ID NO:2.

As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity. more preferably 85%, 95%, or 98% identity are defined herein as sufficiently identical.

As used interchangeably herein a “CARD-4 activity”, “biological activity of CARD-4” or “functional activity of CARD-4”. refers to an activity exerted by a CARD-4 protein, polypeptide or nucleic acid molecule on a CARD-4 responsive cell as determined in vivo, or in vitro, according to standard techniques. A CARD-4 activity can be a direct activity, such as an association with or an enzymatic activity on a second protein or an indirect activity, such as a cellular signaling activity mediated by interaction of the CARD-4 protein with a second protein. In one embodiment, a CARD-4 activity includes at least one or more of the following activities: (1) the ability to form protein:protein interactions with proteins in the apoptotic signalling pathway; (2) the ability to form CARD-CARD interactions with proteins in the apoptotic signaling pathway; (3) the ability to bind a CARD-4 ligand (e.g., CARD-4, CARD-3, caspase 9, and/or BCLX); (4) the ability to bind to an intracellular target; (5) the ability to enhance caspase 9 activity; and (6) the ability to activate the NF-κB pathway. Other activities include: (1) modulation of cellular proliferation; (2) modulation of cellular differentiation; (3) modulation of cellular death; and (4) modulation of inflammation and/or an innate immune response.

I. Isolated Nucleic Acid Molecules

One aspect of the invention pertains to methods of using isolated nucleic acid molecules that encode CARD-4 proteins or biologically active portions thereof, as well as nucleic acid molecules sufficient for use as hybridization probes to identify CARD-4-encoding nucleic acids (e.g., CARD-4 mRNA) and fragments for use as PCR primers for the amplification or mutation of CARD-4 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated CARD-4 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule, e.g., a nucleic acid molecule having the nucleotide sequence of SEQ ID NO:1, SEQ ID NO:3, or a complement of any of these nucleotide sequences, can be isolated using standard molecular biology techniques and the sequence information provided herein. Using all or portion of the nucleic acid sequences of SEQ ID NO:1 or SEQ ID NO:3 as a hybridization probe, CARD-4 nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to CARD-4 nucleotide sequences can be prepared by standard synthetic techniques, e.g., using an automated DNA synthesizer.

In another embodiment, an isolated nucleic acid molecule comprises a nucleic acid molecule which is a complement of the nucleotide sequence shown in SEQ ID NO:1, SEQ ID NO:3, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, the nucleic acid molecules can comprise only a portion of a nucleic acid sequence encoding CARD-4, for example, a fragment which can be used as a probe or primer or a fragment encoding a biologically active portion of CARD-4. The nucleotide sequence determined from the cloning of the human CARD-4 allows for the generation of probes and primers designed for use in identifying and/or cloning CARD-4 homologues in other cell types, e.g., from other tissues, as well as CARD-4 homologues and orthologs from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75. 100, 125, 150, 175, 200, 250, 300, 350 or 400 consecutive nucleotides of the sense or anti-sense sequence of SEQ ID NO:1, SEQ ID NO:3, or of a naturally occurring mutant of one of SEQ ID NO:1 or SEQ ID NO:3.

Probes based on the CARD-4 nucleotide sequence can be used to detect transcripts or genomic sequences encoding the same or similar proteins. The probe comprises a label group attached thereto, e.g., a radioisotope, a fluorescent compound, an enzyme, or an enzyme co-factor. Such probes can be used as a part of a diagnostic test kit for identifying allelic variants and orthologs of the CARD-4 proteins of the present invention, identifying cells-or tissue which mis-express a CARD-4 protein, such as by measuring a level of a CARD-4-encoding nucleic acid in a sample of cells from a subject, e.g., detecting CARD-4 mRNA levels or determining whether a genomic CARD-4 gene has been mutated or deleted.

A nucleic acid fragment encoding a “biologically active portion” of CARD-4 can be prepared by isolating a portion of SEQ ID NO:1 or SEQ ID NO:3 which encodes a polypeptide having a CARD-4 biological activity, expressing the encoded portion of CARD-4 protein (e.g., by recombinant expression in vitro) and assessing the activity of the encoded portion of CARD-4. For example, a nucleic acid fragment encoding a biologically active portion of CARD-4 can include a CARD, NBS, or leucine rich repeat domain. A biologically active portion of CARD-4 can optionally bind caspase 9, enhance caspase 9 activity, activate the NF-κB pathway, and/or activate JNK.

The invention further encompasses nucleic acid molecules that differ from the nucleotide sequence of SEQ D NO:1 or SEQ ID NO:3 due to degeneracy of the genetic code and thus encode the same CARD-4 protein as that encoded by the nucleotide sequence shown in SEQ ID NO:1 or SEQ ID NO:3.

In addition to the CARD-4 nucleotide sequence shown in SEQ ID NO:1 or SEQ D NO:3, it will be appreciated by those skilled in the art that DNA sequence polymorphisms that lead to changes in the amino acid sequences of CARD-4 may exist within a population (e.g., the human population). Such genetic polymorphism in the CARD-4 gene may exist among individuals within a population due to natural allelic variation. As used herein, the terms “gene” and “recombinant gene” refer to nucleic acid molecules comprising an open reading frame encoding a CARD-4 protein, preferably a mammalian CARD-4 protein. Such natural allelic variations can typically result in 1-5% variance in the nucleotide sequence of the CARD-4 gene. Any and all such nucleotide variations and resulting amino acid polymorphisms in CARD-4 that are the result of natural allelic variation and that do not alter the functional activity of CARD-4 are intended to be within the scope of the invention. Thus, e.g., 1%, 2%, 3%, 4%, or 5% of the amino acids in CARD-4 are replaced by another amino acid, preferably the amino acids are replaced by conservative substitutions.

Moreover, nucleic acid molecules encoding CARD-4 proteins from other species (CARD-4 orthologs/homologues), which have a nucleotide sequence which differs from that of a CARD-4 disclosed herein, are intended to be within the scope of the invention.

In general, an allelic variant of a gene will be readily identifiable as mapping to the same chromosomal location as said gene. Allelic variants of human CARD-4 will be readily identifiable as mapping to the human CARD-4 locus on chromosome 7 near genetic marker SHGC-31928.

Accordingly, in another embodiment, an isolated nucleic acid molecule is at least 300 (325, 350, 375, 400, 425, 450, 500, 550, 600, 650, 700, 800, 900, 1000, 1300, 1600 or 1931) nucleotides in length and hybridizes under stringent conditions to the nucleic acid molecule comprising the nucleotide sequence, preferably the coding sequence, of SEQ D NO:1 or SEQ ID NO:3.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the an and can be found in Current Protocols in Molecular Biology. John Wilev & Sons, N.Y. (1989). 6.3.1-6.3.6. An, non-limiting example of stringent hybridization conditions are hybridization in 6× sodium chlorideisodium citrate (SSC) at about 45° C., followed by one or more washes in 0.2×SSC. 0.1% SDS at 50-65° C. (e.g., 50° C. or 60° C. or 65° C.). Preferably, the isolated nucleic acid molecule of the invention that hybridizes under stringent conditions corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the CARD-4 sequence that may exist in the population, the skilled artisan will further appreciate that changes can be introduced by mutation into the nucleotide sequence of SEQ ID NO:1 or SEQ ID NO:3 thereby leading to changes in the amino acid sequence of the encoded protein without altering the functional ability of the protein. For example. one can make nucleotide substitutions leading to amino acid substitutions at “non-essential” amino acid residues. A “non-essential” amino acid residue is a residue that can be altered from the wild-type sequence of CARD-4 without altering the biological activity, whereas an “essential” amino acid residue is required for biological activity. For example, amino acid residues that are conserved among the CARD-4 proteins of various species are predicted to be particularly unamenable to alteration.

For example, preferred CARD-4 proteins of the present invention, contain at least one CARD domain. Additionally, a protein also contains at least one kinase domain or at least one linker domain. A CARD domain contains at least one nucleotide binding domain or Leucine-rich repeats. Such conserved domains are less likely to be amenable to mutation. Other amino acid residues, however, (e.g., those that are not conserved or only semi-conserved among CARD-4 of various species) may not be essential for activity and thus are likely to be amenable to alteration.

Accordingly, another aspect of the invention pertains to nucleic acid molecules encoding CARD-4 proteins that contain changes in amino acid residues that are not essential for activity. Such CARD-4 proteins differ in amino acid sequence from SEQ ID NO:2 and yet retain biological activity. In one embodiment, the isolated nucleic acid molecule includes a nucleotide sequence encoding a protein that includes an amino acid sequence that is at least about 45% identical, 65%, 75%, 85%, 95%, or 98% identical to the amino acid sequence of SEQ ID NO:2.

An isolated nucleic acid molecule encoding a CARD-4 protein having a sequence which differs from that of SEQ ID NO:1 can be created by introducing one or more nucleotide substitutions, additions or deletions into the nucleotide sequence of CARD-4 (SEQ ID NO:1 or SEQ ID NO:3) such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. Thus, for example, 1%, 2%. 3%, 5%, or 10% of the amino acids can be replaced by conservative substitution. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic-side chains (e.g., tyrosine, phenylalanine, tryptophan, histidine). Thus, a predicted nonessential amino acid residue in CARD-4 is preferably replaced with another amino acid residue from the same side chain family. Alternatively, mutations can be introduced randomly along all or part of a CARD-4 coding sequence, such as by saturation mutagenesis, and the resultant mutants can be screened for CARD-4 biological activity to identify mutants that retain activity. Following mutagenesis, the encoded protein can be expressed recombinantly and the activity of the protein can be determined. Preferred mutants possess at least one biological activity possessed by naturally occurring human CARD-4, e.g., (1) the ability to form protein:protein interactions with proteins in the apoptotic signalling pathway; (2) the ability to form CARD-CARD interactions with proteins in the apoptotic signaling pathway; (3) the ability to bind a CARD-4 ligand (e.g., CARD-4, CARD-3, caspase 9, and/or BCLX); (4) the ability to bind to an intracellular target; (5) the ability to enhance caspase 9 activity; and (6) the ability to activate the NF-κB pathway. Other activities include: (1) modulation of cellular proliferation; (2) modulation of cellular differentiation; (3) modulation of cellular death; and (4) modulation of inflammation and/or an innate immune response.

The present invention encompasses antisense nucleic acid molecules, i.e., molecules which are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire CARD-4 coding strand, or to only a portion thereof, e.g., all or part of the protein coding region (or open reading frame). An antisense nucleic acid molecule can be antisense to a noncoding region of the coding strand of a nucleotide sequence encoding CARD-4. The noncoding regions (“5′ and 3′ untranslated regions”) are the 5′ and 3′ sequences which flank the coding region and are not translated into amino acids. Given the coding strand sequences encoding CARD-4 disclosed herein, antisense nucleic acids of the invention can be designed according to the rules of Watson and Crick base pairing. The antisense nucleic acid molecule can be complementary to the entire coding region of CARD-4 mRNA, but more preferably is an oligonucleotide which is antisense to only a portion of the coding or noncoding region of CARD-4 mRNA. An antisense oligonucleotide can be, for example. about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil. 2-thiouracil, 4-thiouracil, 5-methyluracil, uracil-5-oxyacetic acid methylester, uracil-5-oxyacetic acid (v), 5-methyl-2-thiouracil, 3-(3-aino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that thev hybridize with or bind to cellular mRNA and/or genomic DNA encoding a CARD-4 protein to thereby inhibit expression of the protein, e.g., by inhibiting transcription and/or translation. The hybridization can be by conventional nucleotide complementarity to form a stable duplex, or, for example. in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (198?) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic ASNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 33 4:585-591)) can be used to catalytically cleave CARD-4 mRNA transcripts to thereby inhibit translation of CARD-4 mRNA. A ribozyme having specificity for a CARD-4-encoding nucleic acid can be designed based upon the nucleotide sequence of a CAR-4 cDNA disclosed herein. For example, a derivative of a Tetrahymena L-19 IVS RNA can be constructed in which the nucleotide sequence of the active site is complementary to the nucleotide sequence to be cleaved in a CARD-4-encoding mRNA. See. e.g., Cech et al. U.S. Pat. No. 4,987,071, and Cech et al. U.S. Pat. No. 5,116,742. Alternatively, CARD-4 mRNA can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, CARD-4 gene expression can be inhibited by targeting nucleotide sequences complementary to the regulatory region of the CARD-4 (e.g., the CARD-4 promoter and/or enhancers) to form triple helical structures that prevent transcription of the CARD-4 gene in target cells. See generally, Helene (1 991) Anticancer Drug Des. 6(6):569-84; Helene (1992) Ann. N.Y. Acad. Sci. 660:27-36. and Maher (1992) Bioassays 14(12):807-15.

In embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93:14670-675.

PNAs of CARD-4 can be used for therapeutic and diagnostic applications. For example, PNAs can be used as antisense or antigene agents for sequence-specific modulation of gene expression by, e.g., inducing transcription or translation arrest or inhibiting replication. PNAs of CARD-4 can also be used. e.g., in the analysis of single base pair mutations in a gene by, e.g., PNA directed PCR clamping; as artificial restriction enzymes when used in combination with other enzymes, e.g., S1 nucleases (Hyrup (1996) supra; or as probes or primers for DNA sequence and hybridization (Hyrup (1996) supra; Perry-O'Keefe et al. (1996) Proc. Natl. Acad. Sci. USA 93: 14670-675).

In another embodiment, PNAs of CARD-4 can be modified, e.g., to enhance their stability or cellular uptake, by attaching lipophilic or other helper groups to PNA, by the formation of PNA-DNA chimeras, or by the use of liposomes or other techniques of drug delivery known in the art. For example. PNA-DNA chimeras of CARD-4 can be generated which may combine the advantageous properties of PNA and DNA. Such chimeras allow DNA recognition enzymes. e.g., RNAse H and DNA polmerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) supra). The synthesis of PNA-DNA chimeras can be performed as described in Hyrup (1996) supra and Finn et al. (1996) Nucleic Acids Research 24(17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag et al. (1989) Nucleic Acid Res. 17:5973-88). PNA monomers are then coupled in a stepwise manner to produce a chimeric molecule with a 5′ PNA segment and a 3′ DNA segment (Finn et al. (1996) Nucleic Acids Research 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556: Lemaitre et al. (1987) Proc. Natl. Acad. Sci. USA 84:648-652′ PCT Publication No. WO 88/09810) or the blood-brain barrier (see, e.g., PCT Publication No. W0 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

II. Isolated CARD-4 Proteins and CARD-4 Antibodies.

One aspect of the invention pertains to methods of using CARD-4 proteins, and biologically active portions thereof, as well as polypeptide fragments suitable for use as immunogens to raise anti-CARD-4 antibodies. In one embodiment, native CARD-4 proteins can be isolated from cells or tissue sources by an appropriate purification scheme using standard protein purification techniques. In another embodiment, CARD-4 proteins are produced by recombinant DNA techniques. Alternative to recombinant expression, a CARD-4 protein or polypeptide can be synthesized chemically using standard peptide synthesis techniques.

An “isolated” or “purified” protein or biologically active portion thereof is substantially free of cellular material or other contaminating proteins from the cell or tissue source from which the CARD-4 protein is derived, or substantially free from chemical precursors or other chemicals when chemically synthesized. The language “substantially-free of cellular material” includes preparations of CARD-4 protein in which the protein is separated from cellular components of the cells from which it is isolated or recombinantly produced. Thus, CARD-4 protein that is substantially free of cellular material includes preparations of CARD-4 protein having less than about 30%, 20%, 10%, or 5% (by dry weight) of non-CARD-4 protein (also referred to herein as a “contaminating protein”). When the CARD-4 protein or biologically active portion thereof is recombinantly produced, it is also preferably substantially free of culture medium, i.e., culture medium represents less than about 20%, 10%, or 5% of the volume of the protein preparation. When CARD-4 protein is produced by chemical synthesis, it is preferably substantially free of chemical precursors or other chemicals, i.e., it is separated from chemical precursors or other chemicals which are involved in the synthesis of the protein. Accordingly such preparations of CAR-4 protein have less than about 30%, 20%, 10%, 5% (by dry weight) of chemical precursors or non-CARD-4 chemicals.

Biologically active portions of a CARD-4 protein include peptides comprising amino acid sequences sufficiently identical to or derived from the amino acid sequence of the CARD-4 protein (e.g., the amino acid sequence shown in SEQ ID NO:2), which include less amino acids than the full length CARD-4 protein, and exhibit at least one activity of a CARD-4 protein. Typically, biologically active portions comprise a domain or motif with at least one activity of the CARD-4 protein. A biologically active portion of a CARD-4 protein can be a polypeptide which is, for example, 10, 25, 50, 100 or more amino acids in length. Preferred biologically active polypeptides include one or more identified CARD-4 structural domains, e.g., the CARD domain, NBS domain, or leucine rich repeats. Preferred biologically active polypeptides posses one or more of the following activites: 1) the ability to form protein:protein interactions with proteins in the apoptotic signalling pathway (2) the ability to form CARD-CARD interactions with proteins in the apoptotic signaling pathway; (3) the ability to bind a CARD-4 ligand (e.g., CARD-4, CARD-3, caspase 9, and/or BCLX); (4) the ability to bind to an intracellular target; (5) the ability to enhance caspase 9 activity; and (6) the ability to activate the NF-κB pathway. Other activities include: (1) modulation of cellular proliferation; (2) modulation of cellular differentiation; (3) modulation of cellular death; and (4) modulation of inflammation and/or an innate immune response.

Moreover, other biologically active portions. in which other regions of the protein are deleted, can be prepared by recombinant techniques and evaluated for one or more of the functional activities of a native CARD-4 protein.

CARD-4 protein has the amino acid sequence shown of SEQ ID NO:2. Other useful CARD-4 proteins are substantially identical to SEQ ID NO:2 and retain the functional activity of the protein of SEQ ID NO:2, yet differ in amino acid sequence due to natural allelic variation or mutagenesis.

A useful CARD-4 protein is a protein which includes an amino acid sequence at least about 45%, preferably 55%, 65%, 75%, 85%, 95%, or 99% identical to the amino acid sequence of SEQ ID NO:2 and retains the functional activity of the CARD-4 protein of SEQ ID NO:2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences similar or homologous to CARD-4 nucleic acid molecules of the invention. BLAST protein searches can be performed with the XBLAST program, score=50, wordlength=3 to obtain amino acid sequences homologous to CARD-4 protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically exact matches are counted.

The invention also provides CARD-4 chimeric or fusion proteins. As used herein, a CARD-4 “chimeric protein” or “fusion protein” comprises a CARD-4 polypeptide operatively linked to a non-CARD-4 polypeptide. A “CARD-4 polypeptide” refers to a polypeptide having an amino acid sequence corresponding to all or a portion (preferably a biologically active portion) of a CARD-4, whereas a “non-CARD-4 polypeptide” refers to a polypeptide having an amino acid sequence corresponding to a protein which is not substantially identical to the CARD-4 protein, e.g., a protein which is different from the CARD-4 proteins and which is derived from the same or a different organism. Within the fusion protein, the term “operatively linked” is intended to indicate that the CARD-4 polypeptide and the non-CARD-4 polypeptide are fused in-frame to each other. The heterologous polypeptide can be fused to the N-terminus or C-terminus of the CARD-4 polypeptide.

One useful fusion protein is a GST fusion protein in which the CARD-4 sequences are fused to the C-terminus of the GST sequences. Such fusion proteins can facilitate the purification of recombinant CARD-4. In another embodiment, the fusion protein contains a signal sequence from another protein. In certain host cells (e.g., mammalian host cells), expression and/or secretion of CARD-4 can be increased through use of a heterologous signal sequence. For example, the gp67 secretory sequence of the baculovirus envelope protein can be used as a heterologous signal sequence (Current Protocols in Molecular Biology, Ausubel et al., eds., John Wiley & Sons. 1992). Other examples of eukaryotic heterologous signal sequences include the secretory sequences of melittin and human placental alkaline phosphatase (Stratagene, La Jolla, Calif.). In yet another example, useful prokaryotic heterologous signal sequences include the phoA secretory signal (Molecular cloning, Sambrook et al, second edition, Cold spring harbor laboratory press, 1989) and the protein A secretory signal (Pharmacia Biotech; Piscataway, N.J.).

In yet another embodiment, the fusion protein is a CARD-4-immunoglobulin fusion protein in which all or part of CARD-4 is fused to sequences derived from a member of the immunoglobulin protein family. The CARD-4-immunoglobulin fusion proteins of the invention can be incorporated into pharmaceutical compositions and administered to a subject to inhibit an interaction between a CARD-4 ligand and a CARD-4 protein on the surface of a cell, to thereby suppress CARD-4-mediated signal transduction in vivo. The CARD-4-immunoglobulin fusion proteins can be used to affect the bioavailability of a CARD-4 cognate ligand. Inhibition of CARD-4 ligand/CARD-4 interaction may be useful therapeutically for both the treatment of proliferative and differentiative disorders, as well as modulating (e.g., promoting or inhibiting) cell survival. Inhibition of the interation may be useful for treatment of inflammatory disorders. Moreover, the CARD-4-immunoglobulin fusion proteins of the invention can be used as immunogens to produce anti-CARD-4 antibodies in a subject, to purify CARD-4 ligands and in screening assays to identify molecules which inhibit the interaction of CARD-4 with a CARD-4 ligand.

Preferably, a CARD-4 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see. e.g., Current Protocols in Molecular Biology, Ausubel et al. eds., John Wiley & Sons: 1992). Moreover, many expression vectors are commercially available that already encode a fusion moiety (e.g., a GST polypeptide). A CARD-4-encoding nucleic acid can be cloned into such an expression vector such that the fusion moiety is linked in-frame to the CARD-4 protein.

The present invention also pertains to variants of the CARD-4 proteins which function as either CARD-4 agonists (mimetics) or as CARD-4 antagonists. Variants of the CARD-4 protein can be generated by mutagenesis, e.g., discrete point mutation or truncation of the CARD-4 protein. An agonist of the CARD-4 protein can retain substantially the same, or a subset, of the biological activities of the naturally occurring form of the CARD-4 protein. An antagonist of the CARD-4 protein can inhibit one or more of the activities of the naturally occurring form of the CARD-4 protein by, for example, competitively binding to a downstream or upstream member of a cellular signaling cascade which includes the CARD-4 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the CARD-4 proteins.

Variants of the CARD-4 protein which function as either CARD-4 agonists (mimetics) or as CARD-4 antagonists can be identified by screening combinatorial libraries of mutants, e.g., truncation mutants of the CARD-4 protein for CARD-4 protein agonist or antagonist activity. In one embodiment, a variegated library of CARD-4 variants is generated by combinatorial mutagenesis at the nucleic acid level and is encoded by a variegated gene library. A variegated library of CARD-4 variants can be produced by, for example, enzymatically ligating a mixture of synthetic oligonucleotides into gene sequences such that a degenerate set of potential CARD-4 sequences is expressible as individual polypeptides, or alternatively, as a set of larger fusion proteins (e.g., for phage display) containing the set of CARD-4 sequences therein. There are a variety of methods which can be used to produce libraries of potential CARD-4 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CARD-4 sequences. Methods for synthesizing degenerate oligonucleotides are known in the art (see, e.g., Narang (1983) Tetrahedron 39:3; Itakura et al. (1984) Annnu. Rev. Biochem. 53:323; Itakura et al. (1984) Science 198:1056; Ike et al. (1983) Nucleic Acid Res. 11:477).

Useful fragments of CARD-4. include fragments comprising or consisting of a domain or subdomain described herein, e.g., a CARD domain, NBS domain, or LRR domain.

In addition, libraries of fragments of the CARD-4 protein coding sequence can be used to generate a variegated population of CARD-4 fragments for screening and subsequent selection of variants of a CARD-4 protein. In one embodiment, a library of coding sequence fragments can be generated by treating a double stranded PCR fragment of a CARD-4 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the CARD-4 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CARD-4 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify CARD-4 variants (Arkin and Yourvan (1992) Proc. Natl. Acad. Sci. USA 89:7811-7815; Delgrave et al. (1993) Protein Engineering 6(3):327-331).

An isolated CARD-4 protein, or a portion or fragment thereof, can be used as an immunogen to generate antibodies that bind CARD-4 using standard techniques for polyclonal and monoclonal antibody preparation. The full-length CARD-4 protein can be used or, alternatively, the invention provides antigenic peptide fragments of CARD-4 for use as immunogens. The antigenic peptide of CARD-4 comprises at least 8 (preferably 10, 15, 20, or 30) amino acid residues of the amino acid sequence shown in SEQ ID NO:2 or polypeptides including amino acids 128-139 or 287-298 of SEQ ID NO:2 and encompasses an epitope of CARD-4 such that an antibody raised against the peptide forms a specific immune complex with CARD-4.

Useful antibodies include antibodies which bind to a domain or subdomain of CARD-4 described herein (e.g., a kinase domain, a CARD domain, or a leucine-rich domain).

Preferred epitopes encompassed by the antigenic peptide are regions of CARD-4 that are located on the surface of the protein, e.g., hydrophilic regions. Other important criteria include a preference for a terminal sequence, high antigenic index (e.g., as predicted by Jameson-Wolf algorithm), ease of peptide synthesis (e.g., avoidance of prolines); and high surface probability (e.g., as predicted by the Emini algorithm; FIG. 8 and FIG. 9).

A CARD-4 immunogen typically is used to prepare antibodies by immunizing a suitable subject, (e.g., rabbit, goat, mouse or other mammal) with the immunogen. Akn appropriate immunogenic preparation can contain, for example, recombinantly expressed CARD-4 protein or a chemically synthesized CARD-4 polypeptide. The preparation can further include an adjuvant, such as Freund's complete or incomplete adjuvant, or similar immunostimulatory agent. Immunization of a suitable subject with an immunogenic CARD-4 preparation induces a polyclonal anti-CARD-4 antibody response. For example. polypeptides including amino acids 128-139 or 287-298 of human CARD-4 were conjugated to KLH and the resulting conjugates were used to immunize rabbits and polyclonal antibodies that specifically recognize the two immunogen peptides were generated.

Accordingly, another aspect of the invention pertains to anti-CARD-4 antibodies. The term “antibody” as used herein refers to immunoglobulin molecules and immunologically active portions of immunoglobulin molecules, i.e., molecules that contain an antigen binding site which specifically binds an antigen, such as CARD-4. A molecule which specifically binds to CARD-4 is a molecule which binds CARD-4, but does not substantially bind other molecules in a sample, e.g., a biological sample, which naturally contains CARD-4. Examples of immunologically active portions of immunoglobulin molecules include F(ab) and F(ab′)2 fragments which can be generated by treating the antibody with an enzyme such as pepsin. The invention provides polyclonal and monoclonal antibodies that bind CARD-4. The term “monoclonal antibody” or “monoclonal antibody composition”, as used herein, refers to a population of antibody molecules that contain only one species of an antigen binding site capable of immunoreacting with a particular epitope of CARD-4. A monoclonal antibody composition thus typically displays a single binding affinity for a particular CARD-4 protein with which it immunoreacts.

Polyclonal anti-CARD-4 antibodies can be prepared as described above by immunizing a suitable subject with a CARD-4 immunogen. The anti-CARD-4 antibody titer in the immunized subject can be monitored over time by standard techniques, such as with an enzyme linked immunosorbent assay (ELISA) using immobilized CARD-4. If desired, the antibody molecules directed against CARD-4 can be isolated from the mammal (e.g., from the blood) and fer purified by well-known techniques, such as protein A chromatography to obtain the IgG fraction. At an appropriate time after immunization, e.g., when the anti-CARD-4 antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing various antibodies monoclonal antibody hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with a CARD-4 immunogen as described above, and the culture supernatants of the resulting hybridoma cells are screened to identify a hybridoma producing a monoclonal antibody that binds CARD-4.

Any of the many well known protocols used for fusing lymphocytes and immortalized cell lines can be applied for the purpose of generating an anti-CARD-4 monoclonal antibody (see, e.g., Current Protocols in Immunology, supra: Galfre et al. (1977) Nature 266:55052; R. H. Kenneth. in Monoclonal Antibodies: A New Dimension In Biological Analyses, Plenum Publishing Corp., New York, N.Y. (1980); and Lemer (1981) Yale J. Biol. Med., 54:387-402). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not tansformed). Hybridoma cells producing a monoclonal antibody of the invention are detected by screening the hybridoma culture supematants for antibodies that bind CARD-4, e.g., using a standard ELISA assay.

Alternative to preparing monoclonal antibody-secreting hybridomas, a monoclonal anti-CARD-4 antibody can be identified and isolated by screening a recombinant combinatorial immunoglobulin library (e.g., an antibody phage display library) with CARD-4 to thereby isolate inumunoglobulin library members that bind CARD-4. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library, can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-CARD-4 antibodies, such as chimeric and humanized monoclonal antibodies, comprising both human and non-human portions, which can be made using standard recombinant DNA techniques, are Awithin the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125.023; Better et al. (1988) Science 240:1041-1043; Liu et al. (1987) Proc. Natl. Acad. Sci. USA 84:3439-3443; Liu et al. (1987) J. Inmunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, (1985) Science 229:1202-1207; Oi et al. (1986) Bio/Techniques 4:214; U.S. Patent 5.225,539; Jones et al. (1986) Nature 321:552-525; Verhoevan et al. (1988) Science 239:1534; and Beidler et al. (1988) J. Immunol. 141:4053-4060.

An anti-CARD-4 antibody (e.g., monoclonal antibody) can be used to isolate CARD-4 by standard techniques, such as affinity chromatography or immunoprecipitation. An anti-CARD-4 antibody can facilitate the purification of natural CARDS from cells and of recombinantly produced CARD-4 expressed in host cells. Moreover, an anti-CARD-4 antibody can be used to detect CARD-4 protein (e.g., in a cellular lysate or cell supernatant) in order to evaluate the abundance and pattern of expression of the CARD-4 protein. Anti-CARD-4 antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups. fluorescent materials, luminescent materials, bioluminescent materials. and radioactive materials. Examples of suitable enzymes include horseradish peroxidase, alkaline phosphatase, β-galactosidase, or acetylcholinesterase; examples of suitable prosthetic group complexes include streptavidinfbiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding CARD-4 (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operatively linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., CARD-4 proteins, mutant forms of CARD-4, fusion proteins, etc.).

The recombinant expression vectors of the invention can be designed for expression of CARD-4 in prokaryotic or eukaryotic cells, e.g., bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein: 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40). pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185. Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the CARD-4 expression vector is a yeast expression vector. Examples of vectors for expression in yeast S. cerivisae include pYepSec1 (Baldari et al. (1987) EMBO J. 6:229-234), pMFa (Kuan and Herskowitz, (1982) Cell 30:933-943), pJRY88 (Schultz et al. (1987) Gene 54:113-123), pYES2 (Invitrogen Corporation, San Diego, Calif.), pGBT9 (Clontech, Palo Alto, Calif.), pGAD10 (Clontech, Palo Alto. Calif.). pYADE4 and pYGAE2 and pYPGE2 (Brunelli and Pall, (1993) Yeast 9:1299-1 308). pYPGE15 (Brunelli and Pall, (1993) Yeast 9:1309-1318), pACTII (Dr. S. E. Elledge. Baylor College of Medicine), and picZ (InVitrogen Corp, San Diego, Calif.).

Alternatively, CARD-4 can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840), pCI (Promega), and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomecalovirus and Simian Virus 40. For other suitable expression systems for both prokaryotic and eukaryotic cells see chapters 16 and 17 of Sambrook et al. (supra).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873.316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to CARD-4 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory, region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention or isolated nucleic acid molecule of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, CARD-4 protein can be expressed in bacterial cells such as E. coli, insect cells, yeast or mammalian cells (such as Chinese hamster ovary cells (CHO) or COS cells). Other suitable host cells are known to those skilled in the art.

Vector DNA or an isolated nucleic acid molecule of the invention can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In some cases vector DNA is retained by the host cell. In other cases the host cell does not retain vector DNA and retains only an isolated nucleic acid molecule of the invention carried by the vector. In some cases, and isolated nucleic acid molecule of the invention is used to transform a cell without the use of a vector.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding CARD-4 or can be introduced on a separate vector. Cells stably transfected with the introduced nucleic acid can be identified by drug selection (e.g., cells that have incorporated the selectable marker gene will survive, while the other cells die).

A host cell of the invention. such as a prokaryotic or eukaryotic host cell in culture, can be used to produce (i.e., express) a CARD-4 protein. Accordingly, the invention further provides methods for producing CARD-4 protein using the host cells of the invention. In one embodiment, the method comprises culturing the host cell of the invention (into which a recombinant expression vector or isolated nucleic acid molecule encoding CARD-4 has been introduced) in a suitable medium such that CARD-4 protein is produced. In another embodiment, the method further comprises isolating CARD-4 from the medium or the host cell.

The host cells of the invention can also be used to produce nonhuman transgenic animals. For example, in one embodiment, a host cell of the invention is a fertilized oocyte or an embryonic stem cell into which CARD-4-coding sequences have been introduced. Such host cells can then be used to create non-human transgenic animals in which exogenous CARD-4 sequences have been introduced into their genome or homologous recombinant animals in which endogenous CARD-4 sequences have been altered. Such animals are useful for studying the function and/or activity of CARD-4 and for identifying and/or evaluating modulators of CARD-4 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous CARD-4 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing CARD-4-encoding nucleic acid into the male pronuclei of a fertilized oocyte. e.g., by microinjection, retroviral infection, and allowing the oocyte to develop in a pseudopregnant female foster animal. The CARD-4 cDNA sequence. e.g., that of SEQ ID NO:1 or SEQ ID NO:3 can be introduced as a transgene into the genome of a non-human animal. Alternatively a nonhuman homolog or ortholog of the human CARD-4 gene, such as a mouse CARD-4 gene, can be isolated based on hybridization to the human CARD-4 cDNA and used as a transgene. For example, the mouse ortholog of CARD-4 can be used to make a transgenic animal using standard methods. Intronic sequences and polyadenylation signals can also be included in the transgene to increase the efficiency of expression of the transgene. A tissue-specific regulatory sequence(s) can be operably linked to the CARD-4 transgene to direct expression of CARD-4 protein to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the CARD-4 transgene in its genome and/or expression of CARD-4 mRNA in tissues or cells of the animals. A transgenic founder animal can then be used to breed additional animals carrying the transgene. Moreover, transgenic animals carrying a transgene encoding CARD-4 can further be bred to other transgenic animals carrying other transgenes.

To create an homologous recombinant animal, a vector is prepared which contains at least a portion of a CARD-4 gene (e.g., a human or a non-human homolog of the CARD-4 gene, e.g., a munine CARD-4 gene) into which a deletion, addition or substitution has been introduced to thereby alter, e.g., functionally disrupt, the CARD-4 gene. In an embodiment, the vector is designed such that, upon homologous recombination, the endogenous CARD-4 gene is functionally disrupted (i.e., no longer encodes a functional protein; also referred to as a “knock out” vector). Alternatively, the vector can be designed such that, upon homologous recombination, the eridogenous CARD-4 gene is mutated or otherwise altered but still encodes functional protein (e.g., the upstream regulatory region can be altered to thereby alter the expression of the endogenous CARD-4 protein). In the homologous recombination vector, the altered portion of the CARD-4 gene is flanked at its 5′ and 3′ ends by additional nucleic acid of the CARD-4 gene to allow for homologous recombination to occur between the exogenous CARD-4 gene carried by the vector and an endogenous CARD-4 gene in an embryonic stem cell. The additional flanking CARD-4 nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced CARD-4 gene has homologously recombined with the endogenous CARD-4 gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 1I13-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The sequence of a nucleic acid encoding murine CARD-4, which can be used in the preparation of a transgenic animal or a homologous recombinant animal, is described in U.S. Pat. No. 6,369,196, the content of which is incorporated herein by reference.

IV. Pharmaceutical Compositions

The CARD-4 nucleic acid molecules, CARD-4 proteins, and anti-CARD-4 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an acent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity may, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular at eight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to modulated. Such appropriate doses may be determined using the assays described herein. When one or more of these small moleucles is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL? (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a CARD-4 protein or anti-CARD-4 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydnrdes, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body w eight (generally 10 mg/kg to 20 mg/kg). If the antibody is to act in the brain, a dosage of 50 mg,kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials, and pharmacogenomics); and d) methods of treatment (e.g., therapeutic and prophylactic). A CARD-4 protein interacts with other cellular proteins and can thus be used for (i) regulation of cellular proliferation; (ii) regulation of cellular differentiation; and (iii) regulation of cell survival. The isolated nucleic acid molecules of the invention can be used to express CARD-4 protein (e.g., via a recombinant expression vector in a host cell in gene therapy, applications), to detect CARD-4 mRNA (e.g., in a biological sample) or a genetic lesion in a CARD-4 gene, and to modulate CARD-4 activity. In addition, the CARD-4 proteins can be used to screen drugs or compounds which modulate the CARD-4 activity or expression as well as to treat disorders characterized by insufficient or excessive production of CARD-4 protein or production of CARD-4 protein forms which have decreased or aberrant activity compared to CARD-4 wild type protein. In addition, the anti-CARD-4 antibodies of the invention can be used to detect and isolate CARD-4 proteins and modulate CARD-4 activity.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides. peptidomimetics, small molecules or other drugs) which bind to CRD proteins or biologically active portions thereof or have a stimulator, or inhibitory effect on, for example, CARD-4 expression or CARD-4 activity. In example of a biologically active portion of human CARD-4 is amino acids 1-145 encoding the CARD domain or amino acids 406-953 of human CARD-4 comprising the leucine rich repeat domain.

Among the screening assays provided by the invention are screening to identify molecules that prevent the dimerization of a CARD-containing polypeptide of the invention, screening to identify molecules which block the binding of a CARD containing polypeptide to a CARD-containing polypeptide of the invention (e.g., CARD-4), screening to identify a competitive inhibitor of the binding of a nucleotide to the nucleotide binding site of a CARD-containing polypeptide of the invention. e.g., human CARD-4, screening to identify compounds which block the interaction between the leucine-rich repeat of a CARD-containing polypeptide of the invention and a ligand which binds to the leucine-rich repeat.

In one embodiment, the invention provides assays for screening candidate or test compounds which bind to or modulate the activity of a CARD-4 proteins or polypeptides or biologically active portions thereof. The activites that can be screened for include one or more of: 1) the ability to form protein:protein interactions with proteins in the apoptotic signalling pathway; (2) the ability to form CARD-CARD interactions with proteins in the apoptotic signaling pathway, (3) the ability to bind a CARD-4 ligand (e.g., CARD4, CARD-3, caspase 9, and/or BCLX); (4) the ability to bind to an intracellular target, (5) the ability to enhance caspase 9 activity; and (6) the ability to activate the NF-κB pathway. Other activities include: (1) modulation of cellular proliferation; (2) modulation of cellular differentiation; (3) modulation of cellular death; and (4) modulation of inflammation and/or an innate immune response.

The test compounds of the present invention can be obtained using any of the numerous approaches in combinatorial library methods known in the art, including: biological libraries; spatially addressable parallel solid phase or solution phase libraries: synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angers. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5.223,409). spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

Determining the ability of the test compound to modulate the activity of CARD-4 or a biologically active portion thereof can be accomplished, for example, by determining the ability of the CARD-4 protein to bind to or interact with a CARD-4 target molecule. As used herein, a “target molecule” is a molecule with which a CARD-4 protein binds or interacts in nature, for example, a molecule associated with the internal surface of a cell membrane or a cytoplasmic molecule. A CARD-4 target molecule can be a non-CARD-4 molecule or a CARD-4 protein or polypeptide of the present invention. In one embodiment, a CARD-4 target molecule is a component of an apoptotic signal transduction pathway, e.g., CARD-4. The target, for example, can be a second intracellular protein which has catalytic activity or a protein which facilitates the association of downstream signaling molecules with CARD-4.

Determining the ability of the test compound to modulate the activity of CARD-4 or a biologically active portion thereof can be accomplished, for example, by determining the ability of the CARD-4 protein to bind to or interact with any of the specific proteins listed in the previous paragraph as CARD-4 target molecules. In another embodiment, CARD-4 target molecules include all proteins that bind to a CARD-4 protein or a fragment thereof in a two-hybrid system binding assay which can be used without undue experimentation to isolate such proteins from cDNA or genomic two-hybrid system libraries. The binding assays described in this section can be cell-based or cell free (described subsequently).

Determining the ability of the CARD-4 protein to bind to or interact with a CARD-4 target molecule can be accomplished by one of the methods described above for determining direct binding. In an embodiment, determining the ability of the CARD-4 protein to bind to or interact with a CARD-4 target molecule can be accomplished by determining the activity of the target molecule. For example, the activity of the target molecule can be determined by detecting induction of a cellular second messenger of the target (e.g., intracellular Ca2+, diacylglycerol, IP3, etc.), detecting catalytic/enzymatic activity of the target on an appropriate substrate, detecting the induction of a reporter gene (e.g., a CARD-4-responsive regulatory element operatively linked to a nucleic acid encoding a detectable marker, e.g. luciferase), or detecting a cellular response, for example, cell survival, cellular differentiation, or cell proliferation. Because CARD-4 enhances caspase 9 activity, activity can be monitored by assaying the caspase 9-mediated apoptosis cellular response or caspase 9 enzymatic activity. In addition, and in another embodiment, genes induced by CARD-4 expression can be identified by expressing CARD-4 in a cell line and conducting a transcriptional profiling experiment wherein the mRNA expression patterns of the cell line transformed with an empty expression vector and the cell line transformed with a CARD-4 expression vector are compared. The promoters of genes induced by CARD-4 expression can be operatively linked to reporter genes suitable for screening such as luciferase, secreted alkaline phosphatase, or beta-galactosidase and the resulting constructs could be introduced into appropriate expression vectors. A recombinant cell line containing CARD-4 and transfected with an expression vector containing a CARD-4 responsive promoter operatively linked to a reporter gene can be used to identify test compounds that modulate CARD-4 activity by assaying the expression of the reporter gene in response to contacting the recombinant cell line with test compounds. CARD-4 agonists can be identified as increasing the expression of the reporter gene and CARD-4 antagonists can be identified as decreasing the expression of the reporter gene.

In another embodiment of the invention, the ability of a test compound to modulate the activity of CARD-4, or biologically active portions thereof can be determined by assaying the ability of the test compound to modulate CARD-4-dependent pathways or processes where the CARD-4 target proteins that mediate the CARD-4 effect are known or unknown. Potential CARD-4-dependent pathways or processes include, but are not limited to, the modulation of cellular signal transduction pathways and their related second messenger molecules (e.g., intracellular Ca²⁻, diacylglycerol, IP3, cAMP etc.), cellular enzymatic activities, cellular responses (e.g., cell survival, cellular differentiation, or cell proliferation), or the induction or repression of cellular or heterologous mRNAs or proteins. CARD-4-dependent pathways or processes could be assayed by standard cell-based or cell free assays appropriate for the specific pathway or process under study. In another embodiment, cells cotransfected with CARD-4 and the NF-κB luciferase reporter gene could be contacted with a test compound and test compounds that block CARD-4 activity could be identified by their reduction of CARD-4-dependent NF-κB pathway lutciferase reporter gene expression. Test compounds that agonize CARD-4 would be expected to increase reporter gene expression. In another embodiment, CARD-4 could be expressed in a cell line and the recombinant CARD-4-expressing cell line could be contacted with a test compound. Test compounds that inhibit CARD-4 activity could be indentified by their reduction of CARD-4-depended NF-κB pathway stimulation as measured by the assay of a NF-κB pathway reporter gene, NF-κB nuclear localization, IκB phosphorylation or proteolysis, or other standard assays for NF-κB pathway activation known to those skilled in the art. In yet another embodiment, assay of the present invention is a cell-free assay comprising contacting a CARD-4 protein or biologically active portion thereof with a test compound and determining the ability of the test compound to bind to the CARD-4 protein or biologically active portion thereof Binding of the test compound to the CARD-4 protein can be determined either directly or indirectly as described above. In one embodiment, a competitive binding assay includes contacting the CARD-4 protein or biologically active portion thereof with a compound known to bind CARD-4 to form an assay mixture, contacting the assay mixture with a test compound, and determining the ability of the test compound to interact with a CARD-4 protein, wherein determining the ability of the test compound to interact with a CARD-4 protein comprises determining the ability of the test compound to preferentially bind to CARD-4 or biologically active portion thereof as compared to the known binding compound.

In another embodiment, an assay is a cell-free assay comprising contacting CARD-4 protein or biologically active portion thereof with a test compound and determining the ability of the test compound to modulate (e.g., stimulate or inhibit) the activity of the CARD-4 protein or biologically active portion thereof Determining the ability of the test compound to modulate the activity of CARD-4 can be accomplished, for example, by determining the ability of the CARD-4 protein to bind to a CARD-4 target molecule by one of the methods described above for determining direct binding. In an alternative embodiment, determining the ability of the test compound to modulate the activity of CARD-4 can be accomplished by determining the ability of the CARD-4 protein to further modulate a CARD-4 target molecule. For example, the catalytic/enzymatic activity of the target molecule on an appropriate substrate can be determined as previously described.

In yet another embodiment, the cell-free assay comprises contacting the CARD-4 protein or biologically active portion thereof with a known compound which binds CARD-4 to form an assay mixture, contacting the assay mixture with a test compound, and determining the ability of the test compound to interact with a CARD-4 protein, wherein determining the ability of the test compound to interact with a CARD-4 protein comprises determining the ability of the CARD-4 protein to preferentially bind to or modulate the activity of a CARD-4 target molecule. The cell-free assays of the present invention are amenable to use of both the soluble form or the membrane-associated form of CARD-4. A membrane-associated form of CARD-4 refers to CARD-4 that interacts with a membrane-bound target molecule. In the case of cell-free assays comprising the membrane-associated form of CARD-4, it may be desirable to utilize a solubilizing agent such that the membrane-associated form of CARD-4 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton□ X-100, Triton□ X-14, Thesit□, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-propane sulfonate (CHAPSO), or N-dodecyl=N,N-dimethyl-3-ammonio-1-propane sulfonate.

In more than one embodiment of the above assay methods of the present invention, it may be desirable to immobilize either CARD-4 or its target molecule to facilitate separation of complexed from uncomplexed forms of one or both of the proteins, as well as to accommodate automation of the assay. Binding of a test compound to CARD-4, or interaction of CARD-4 with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/CARD-4 fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or CARD-4 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of CARD-4 binding or activity determined using standard techniques. In an alternative embodiment. MYC or HA epitope tag CARD-4 fusion proteins or MYC or HA epitope tag target fusion proteins can be adsorbed onto anti-MYC or anti-HA antibody coated microbeads or onto anti-MYC or anti-HA antibody coated microtitre plates, which are then combined with the test compound or the test compound and either the non-adsorbed target protein or CARD-4 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of CARD-4 binding or activity determined using standard techniques.

Other techniques for immobilizing proteins on matrices can also be used in the screening assays of the invention. For example, CARD-4 or its target molecule can be immobilized utilizing conjugation of biotin and streptavidin. Biotinylated CARD-4 or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with CARD-4 or target molecules but which do not interfere with binding of the protein to its target molecule can be derivatized to the wells of the plate, and unbound target or protein trapped in the wells by antibody conjugation. Methods for detecting such complexes, in addition to those described above for the GST-immobilized complexes and epitope tag immobilized complexes, include immunodetection of complexes using antibodies reactive with the CARD-4 or target molecule, as well as enzyme-linked assays which rely on detecting an enzymatic activity associated with the CARD-4 or target molecule.

In another embodiment, modulators of CARD-4 expression are identified in a method in which a cell is contacted with a candidate compound and the expression of the CARD-4 promoter, mRNA or protein in the cell is determined. The level of expression of CARD-4 mRNA or protein in the presence of the candidate compound is compared to the level of expression of CARD-4 mRNA or protein in the absence of the candidate compound. The candidate compound can then be identified as a modulator of CARD-4 expression based on this comparison. For example, when expression of CARD-4 mRNA or protein is greater (statistically significantly greater) in the presence of the candidate compound than in its absence, the candidate compound is identified as a stimulator of CARD-4 mRNA or protein expression. Alternatively, when expression of CARD-4 mRNA or protein is less (statistically significantly less) in the presence of the candidate compound than in its absence, the candidate compound is identified as an inhibitor of CARD-4 mRNA or protein expression. The level of CARD-4 mRNA or protein expression in the cells can be determined by methods described herein for detecting CARD-4 mRNA or protein. The activity of the CARD-4 promoter can be assayed by linking the CARD-4 promoter to a reporter gene such as luciferase, secreted alkaline phosphatase, or beta-galactosidase and introducing the resulting construct into an appropriate vector, transfecting a host cell line, and measuring the activity of the reporter gene in response to test compounds.

In yet another aspect of the invention, the CARD-4 proteins can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with CARD-4 (“CARD-4-binding proteins” or “CARD-4-bp”) and modulate CARD-4 activity. Such CARD-4-binding proteins are also likely to be involved in the propagation of signals by the CARD-4 proteins as, for example, upstream or downstream elements of the CARD-4 pathway.

The two-hybrid system is based on the modular nature of most transcription factors, which consist of separable DNA-binding and activation domains. Briefly, the assay utilizes two different DNA constructs. In one construct, the gene that codes for CARD-4 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an CARD-4-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with CARD-4.

In an embodiment of the invention, the ability of a test compound to modulate the activity of CARD-4, or a biologically active portion thereof can be determined by assaying the ability of the test compound to block the binding of CARD-4 to its target proteins in a two-hybrid system assay. To screen for test compounds that block the interaction between CARD-4 and their target proteins, a yeast two-hybrid screening strain coexpressing the interacting bait and prey constructs, for example, a CARD-4 bait construct, is contacted with the test compound and the activity of the two-hybrid system reporter gene, usually HIS3, lacZ, or URA3 is assayed. If the strain remains viable but exhibits a significant decrease in reporter gene activity, this would indicate that the test compound has inhibited the interaction between the bait and prey proteins. This assay could be automated for high throughput drug screening purposes. In another embodiment of the invention, CARD-4 and their target proteins could be configured in the reverse two-hybrid system (Vidal et al. (1996) Proc. Natl. Acad. Sci. USA 93:10321-6 and Vidal et al. (1996) Proc. Natl. Acad. Sci. USA 93:10315-20) designed specifically for efficient drug screening. In the reverse two-hybrid system, inhibition of a CARD-4 physical interaction with a target protein would result in induction of a reporter gene in contrast to the normal two-hybrid system where inhibition of CARD-4 physical interaction with a target protein would lead to reporter gene repression. The reverse two-hybrid system is preferred for drug screening because reporter gene induction is more easily assayed than report gene repression.

Alternative embodiments of the invention are proteins found to physically interact with proteins that bind to CARD-4. CARD-4 interactors, including but not limited to hNUDC and CARD-3, could be configured into two-hybrid system baits and used in two-hybrid screens to identify additional members of the CARD-4 pathway. The interactors of CARD-4 interactors identified in this way could be useful targets for therapeutic intervention in CARD-4 related diseases and pathologies and an assay of their enzymatic or binding activity could be useful for the identification of test compounds that modulate CARD-4 activity.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, CARD-4 nucleic acid molecules described herein or fragments thereof, can be used to map the location of CARD-4 genes on a chromosome. The mapping of the CARD-4 sequences to chromosomes is an important first step in correlating these sequences with genes associated with disease.

Briefly, CARD-4 genes can be mapped to chromosomes by preparing PCR primers (preferably 15-25 bp in length) from the CARD-4 sequences. Computer analysis of CARD-4 sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the CARD-4 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the CARD-4 sequences to design oligonucleotide primers, sublocalization can be achieved with panels of fragments from specific chromosomes. Other mapping strategies which can similarly be used to map a CARD-4 sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of bindina to a unique chromosomal location with sufficient signal intensity for simple detection. Preferably 1,000 bases, and more preferably 2,000 bases will suffice to get good results at a reasonable amount of time. For a review of this technique, see Verma et al., (Human Chromosomes: A Manual of Basic Techniques (Pergamon Press, New York. 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick. Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature, 325:783-787.

Moreover, differences in the DNA sequences between individuals affected and unaffected with a disease associated with the CARD-4 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The CARD-4 sequences of the present invention can also be used to identify individuals from minute biological samples. The United States military, for example. is considering the use of restriction fragment length polymorphism (RFLP) for identification of its personnel. In this technique, an individual's zenomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the CARD-4 sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The CARD-4 sequences of the invention uniquely represent portions of the human Penome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from CARD-4 sequences described herein is used to generate a unique identification database for an individual, those same reagents can later be used to identify tissue from that individual. Using the unique identification database, positive identification of the individual, living or dead, can be made from extremely small tissue samples.

3. Use of Partial Sequences in Forensic Biology

DNA-based identification techniques can also be used in forensic biology. Forensic biology is a scientific field employing genetic typing of biological evidence found at a crime scene as a means-for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the CARD-4 sequences or portions thereof, e.g., fragments derived from the noncoding regions of SEQ ID NO:1 which have a length of at least 20 or 30 bases.

The sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such CARD-4 probes can be used to identify tissue by species and/or by organ type.

In a similar fashion, these reagents, e.g., CARD-4 primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trials are used for prognostic predictive) purposes to thereby treat an individual prophylactically. Accordingly, one aspect of the present invention relates to diagnostic assays for determining CARD-4 protein and/or nucleic acid expression as well as CARD-4 activity, in the context of a biological sample (e.g., blood, serum, cells, tissue) to thereby determine whether an individual is afflicted with a disease or disorder, or is at risk of developing a disorder, associated with aberrant CARD-4 expression or activity. The invention also provides for prognostic (or predictive) assays for determining whether an individual is at risk of developing a disorder associated Awith CARD-4 protein, nucleic acid expression or activity. For example, mutations in a CARD-4 gene can be assayed in a biological sample. Such assays can be used for prognostic or predictive purpose to thereby prophylactically treat an individual prior to the onset of a disorder characterized by or associated Awith CARD-4 protein, nucleic acid expression or activity.

Another aspect of the invention provides methods for determining CARD-4 protein nucleic acid expression or CARD-4 activity in an individual to thereby select appropriate therapeutic or prophylactic agents for that individual (referred to herein as “pharmacogenomics”). Pharmacogenomics allows for the selection of agents (e.g. drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression or activity of CARD-4 in clinical trials.

These and other agents are described in further detail in the following sections.

1. Diagnostic Assays

An exemplary method for detecting the presence or absence of CARD-4 in a biological sample involves obtaining a biological sample from a test subject and contacting the biological sample with a compound or an agent capable of detecting CARD-4 protein or nucleic acid (e.g., mRNA, genomic DNA) that encodes CARD-4 protein such that the presence of CARD-4 is detected in the biological sample. An agent for detecting CARD-4 mRNA or genomic DNA is a labeled nucleic acid probe capable of hybridizing to CARD-4 mRNA or genomic DNA. The nucleic acid probe can be. for example, a full-length CARD-4 nucleic acid, such as the nucleic acid of SEQ ID NO:1 or 3. or a portion thereof, such as an oligonucleotide of at least 15, 30, 50, 100, 250 or 500 nucleotides in length and sufficient to specifically hybridize under stringent conditions to mRNA or genomic DNA, or a human CARD-4 splice variant such as those described in U.S. Pat. Nos. 6.340,576 or 6.369,196. Other suitable probes for use in the diagnostic assays of the invention are described herein.

An agent for detecting CARD-4 protein can be an antibody capable of binding to CARD-4 protein, preferably an antibody with a detectable label. Antibodies can be polyclonal, or more preferably, monoclonal. For example, polypeptides corresponding to amino acids 128-139 and 287-298 of human CARD-4 have been used to immunize rabbits and produce polyclonal antibodies that specifically recognize human CARD-4. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect CARD-4 mRNA, protein, or genomic DNA in a biological sample in vitro as well as in vivo. For example, in vitro techniques for detection of CARD-4 mRNA include Northern hybridizations and in situ hybridizations. In vitro techniques for detection of CARD-4 protein include enzyme linked immunosorbent assays (ELISAs), Western blots, immunoprecipitations and immunofluorescence. In vitro tehniques for detection of CARD-4 genomic DNA include Southern hybridizatios Furthermore, in vivo techniques for detection of CARD-4 protein include introducing into a subject a labeled anti-CARD-4 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A biological sample is a peripheral blood leukocyte sample isolated by conventional means from a subject.

In another embodiment, the methods further involve obtaining a control biological sample from a control subject, contacting the control sample with a compound or agent capable of detecting CARD-4 protein, mRNA, or genomic DNA, such that the presence of CARD-4 protein, mRNA or genomic DNA is detected in the biological sample, and comparing the presence of CARD-4 protein, mRNA or genomic DNA in the control sample with the presence of CARD-4 protein, mRNA or genomic DNA in the test sample.

The invention also encompasses kits for detecting the presence of CARD-4 in a biological sample (a test sample). Such kits can be used to determine if a subject is suffering from or is at increased risk of developing a disorder associated with aberrant expression of CARD-4 (e.g., an immunological disorder). For example, the kit can comprise a labeled compound or agent capable of detecting CARD-4 protein or mRNA in a biological sample and means for determining the amount of CARD-4 in the sample (e.g., an anti-CARD-D-4 antibody or an oligonucleotide probe which binds to DNA encoding CARD-4. e.g.. Kits may also include instruction for observing, that the tested subject is suffering from or is at risk of developing a disorder associated with aberrant expression of CARD-4 if the amount of CARD-4 protein or mRNA is above or below a normal level.

For antibody-based kits, the kit may comprise, for example: (1) a first antibody (e.g., attached to a solid support) which binds to CARD-4 protein, and, optionally, (2) a second, different antibody which binds to CARD-4 protein or the first antibody and is conjugated to a detectable agent.

For oligonucleotide-based kits, the kit may comprise, for example: (1) a oligonucleotide, e.g., a detectably labelled oligonucleotide, which hybridizes to a CARD-4 nucleic acid sequence or (2) a pair of primers useful for amplifying a CARD-4 nucleic acid molecule.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of CARD-4.

2. Prognostic Assays

The methods described herein can furthermore be utilized as diagnostic or prognostic assays to identify subjects having or at risk of developing a disease or disorder associated with aberrant CARDS expression or activity. For example, the assays described herein, such as the preceding diagnostic assays or the following assays, can be utilized to identify a subject having or at risk of developing a disorder associated with CARD-4 protein, nucleic acid expression or activity. Alternatively, the prognostic assays can be utilized to identify a subject having or at risk for developing such a disease or disorder. Thus, the present invention provides a method in which a test sample is obtained from a subject and CARD-4 protein or nucleic acid (e.g., mRNA, genomic DNA) is detected, wherein the presence of CARD-4 protein or nucleic acid is diagnostic for a subject having or at risk of developing a disease or disorder associated with aberrant CARD-4 expression or activity. As used herein, a “test sample” refers to a biological sample obtained from a subject of interest. For example, a test sample can be a biological fluid (e.g., serum), cell sample, or tissue. Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant CARD-4 expression or activity. For example, such methods can be used to determine whether a subject can be effectively treated with a specific agent or class of agents (e.g., agents of a type which decrease CARD-4 activity). Thus, the present invention provides methods for determining whether a subject can be effectively treated with an agent for a disorder associated with aberrant CARD-4 expression or activity in which a test sample is obtained and CARD-4 protein or nucleic acid is detected (e.g., wherein the presence of CARD-4 protein or nucleic acid is diagnostic for a subject that can be administered the agent to treat a disorder associated with aberrant CARD-4 expression or activity).

The methods of the invention can also be used to detect genetic lesions or mutations in a CARD-4 gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion characterized by at least one of an alteration affecting the integrity of a gene encoding a CARD-4-protein, or the mis-expression of the CARD-4 gene. For example, such genetic lesions can be detected by ascertaining the existence of at least one of 1) a deletion of one or more nucleotides from a CARD-4 gene; 2) an addition of one or more nucleotides to a CARD-4 gene; 3) a substitution of one or more nucleotides of a CARD-4 gene; 4) a chromosomal rearrangement of a CARD-4 gene; 5) an alteration in the level of a messenger RNA transcript of a CARD-4 gene; 6) aberrant modification of a CARD-4 gene, such as of the methylation pattern of the genomic DNA; 7) the presence of a non-wild type splicing pattern of a messenger RNA transcript of a CARD-4 gene (e.g. caused by a mutation in a splice donor or splice acceptor site); 8) a non-wild type level of a CARD-4-protein; 9) allelic loss of a CARD-4 gene; and 10) inappropriate post-translational modification of a CARD-4-protein. As described herein, there are a large number of assay techniques known in the art which can be used for detecting lesions in a CARD-4 gene. A biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the CARD-4-gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a CARD-4 gene under conditions such that hybridization and amplification of the CARD-4-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177). Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a CARD-4 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in CARD-4 can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in CARD-4 can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CARD-4 gene and detect mutations by comparing the sequence of the sample CARD-4 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the CARD-4 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heterodupiexes of formed by hybridizing (labeled) RNA or DNA containing the wild-type CARD-4 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched repions. In other embodiments, either DNA/DNA or RNA/DNA duplexes can be treated w%,ith hydroxylamine or osmium tetroxide and with piperidine in order to digest mismatched regions. After digestion of the mismatched regions, the resulting material is then separated by size on denaturing polyacrylamide gels to determine the site of mutation. See. e.g., Cotton et al (1988) Proc. Natl Acad Sci USA 85:4397; Saleeba et al (1992) Methods Enzymol. 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in CARD-4 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a CARD-4 sequence, e.g., a wild-type CARD-4 sequence, is hybridized to a cDNA or other DNA product from a test cell(s). The duplex is treated with a DNA mismatch repair enzyme, and the cleavage products, if any, can be detected from electrophoresis protocols or the like. See. e.g., U.S. Pat. No. 5,459,039.

In other embodiments, alterations in electrophoretic mobility will be used to identify mutations in CARD-4 genes. For example, single strand conformation polymorphism (SSCP) may be used to detect differences in electrophoretic mobility between mutant and wild type nucleic acids (Orita et al. (1989) Proc Natl. Acad. Sci USA: 86:2766. see also Cotton (1993) Mutat. Res. 285:125-144; and Havashi (1992) Genet Anal Tech Appl 9:73-79). Single-stranded DNA fragments of sample and control CARD-4 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In an embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not corrpletelv denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992),Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used. e.g., in clinical settings to diagnose patients exhibiting symptoms or family history of a disease or illness involving a CARD-4 gene.

Furthermore, any cell type or tissue, preferably peripheral blood leukocytes, in which CARD-4 is expressed may be utilized in the prognostic assays described herein.

3. Pharmacogenomics

Agents, or modulators which have a stimulatory or inhibitory effect on CARD-4 activity (e.g.,CARD-4 gene expression) as identified by a screening assay described herein can be administered to individuals to treat (prophylactically or therapeutically) disorders (e.g., an immunological disorder) associated with aberrant CARD-4 activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of CARD-4 protein, expression of CARD-4 nucleic acid, or mutation content of CARD-4 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of CARD-4 protein, expression of CARD-4 nucleic acid. or mutation content of CARD-4 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a CARD-4 modulator, such as a modulator identified by one of the exemplary screening assays described herein.

4. Monitoring of Effects During Clinical Trials

Monitoring the influence of agents (e.g., drugs, compounds) on the expression or activity of CARD-4 (e.g., the ability to modulate aberrant cell proliferation and/or differentiation) can be applied not only in basic drug screening, but also in clinical trials. For example, the effectiveness of an agent determined by a screening assay as described herein to increase CARD-4 gene expression, protein levels, or upregulate CARD-4 activity, can be monitored in clinical trails of subjects exhibiting decreased CARD-4 gene expression, protein levels, or downregulated CARD-4 activity. Alternatively, the effectiveness of an agent determined by a screening assay to decrease CARD-4 gene expression, protein levels, or downregulated CARD-4 activity, can be monitored in clinical trials of subjects exhibiting increased CARD-4 gene expression, protein levels, or upregulated CARD-4 activity. In such clinical trials, the expression or activity of CARD-4 and, preferably, other genes that have been implicated in, for example. a cellular proliferation disorder can be used as a “read out” or markers of the immune responsiveness of a particular cell.

For example, and not by way of limitation, genes, including CARD-4, that are modulated in cells by treatment with an agent (e.g., compound, drug or small molecule) which modulates CARD-4 activity (e.g., identified in a screening assay as described herein) can be identified. Thus, to study the effect of agents on cellular proliferation disorders, for example, in a clinical trial, cells can be isolated and RNA prepared and analyzed for the levels of expression of CARD-4 and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of CARD-4 or other genes. In this way, the gene expression pattern can serve as a marker, indicative of the physiological response of the cells to the agent. Accordingly, this response state may be determined before, and at various points during, treatment of the individual with the agent.

In an embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a CARD-4 protein, mRNA, or genomic DNA in the preadministration sample; (iii) obtaining one or more post-administration samples from the subject; (iv) detecting the level of expression or activity of the CARD-4 protein, mRNA, or genomic DNA in the post-administration samples; (v) comparing the level of expression or activity of the CARD-4 protein, mRNA, or genomic DNA in the pre-administration sample with the CARD-4 protein, mRNA, or genomic DNA in the post administration sample or samples; and (vi) altering the administration of the agent to the subject accordingly. For example, increased administration of the agent may be desirable to increase the expression or activity of CARD-4 to higher levels than detected, i.e., to increase the effectiveness of the agent. Alternatively, decreased administration of the agent may be desirable to decrease expression or activity of CARD-4 to lower levels than detected, i.e., to decrease the effectiveness of the agent.

5. Transcriptional Profiling

The CARD-4 nucleic acid molecules described herein, including small oligonucleotides, can be used in transcriptionally profiling. For example, these nucleic acids can be used to examine the expression of CARD-4 in normal tissue or cells and in tissue or cells subject to a disease state, e.g., tissue or cells derived from a patient having a disease of interest or cultured cells which model or reflect a disease state of interest, e.g., cells of a cultured tumor cell line. By measuring expression of CARD-4, together or individually, a profile of expression in normal and disease states can be developed. This profile can be used diagnostically and to examine the effectiveness of a therapeutic regime.

C. Methods of Treatment

The present invention provides for both prophylactic and therapeutic methods of treating a subject at risk of (or susceptible to) a disorder or having a disorder associated at with aberrant CARD-4 expression or activity, examples of which are provided herein.

1. Prophylactic Methods

In one aspect, the invention provides a method for preventing in a subject, a disease or condition associated with an aberrant CARD-4 expression or activity, by administering to the subject an agent which modulates CARD-4 expression or at least one CARD-4 activity. Subjects at risk for a disease which is caused or contributed to by aberrant CARD-4, expression or activity can be identified by, for example, any or a combination of diagnostic or prognostic assays as described herein. Administration of a prophylactic agent can occur prior to the manifestation of symptoms characteristic of the CARD-4 aberrancy, such that a disease or disorder is prevented or, alternatively, delayed in its progression. Depending on the type of CARD-4 aberrancy, for example, a CARD-4 agonist or CARD-4 antagonist agent can be used for treating the subject. The appropriate agent can be determined based on screening assays described herein.

2. Therapeutic Methods

Another aspect of the invention pertains to methods of modulating CARD-4 expression or activity for therapeutic purposes. The modulatory method of the invention involves contacting a cell with an agent that modulates one or more of the activities of CARD-4 protein activity associated with the cell. An agent that modulates CARD-4 protein activity can be an agent as described herein, such as a nucleic acid or a protein, a naturally-occurring cognate ligand of a CARD-4 protein, a peptide, a CARD-4 peptidomimetic, or other small molecule. In one embodiment, the agent stimulates one or more of the biological activities of CARD-4 protein. Examples of such stimulatory agents include active CARD-4 protein and a nucleic acid molecule encoding CARD-4 that has been introduced into the cell. In another embodiment, the agent inhibits one or more of the biological activities of CARD-4 protein. Examples of such inhibitory agents include antisense CARD-4 nucleic acid molecules and anti-CARD-4 antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a CARD-4 protein or nucleic acid molecule or a disorder related to CARD-4 expression or activity. In one embodiment, the method involves administering an agent (e.g., an agent identified by a screening assay described herein), or combination of agents that modulates (e.g., upregulates or doamregulates) CARD-4 expression or activity. In another embodiment, the method involves administering a CARD-4 protein or nucleic acid molecule as therapy to compensate for reduced or aberrant CARD-4 expression or activity. Activities of CARD-4 that could be modulated for therapeutic purposes include, but are not limited to: (1) the ability to form protein:protein interactions with proteins in the apoptotic signalling pathway; (2) the ability to form CARD-CARD interactions with proteins in the apoptotic signaling pathway; (3) the ability to bind a CARD-4 ligand (e.g., CARD-4, CARD-3, caspase 9, and/or BCLX); (4) the ability to bind to an intracellular target; (5) the ability to enhance caspase 9 activity and (6) the ability to activate the NF-kB pathway. Other activites that could be modulated for a therapeutic purpose include: (1) modulation of cellular proliferation; (2) modulation of cellular differentiation; (3) modulation of cellular death; (4) modulation of inflammation and/or an innate immune response; and (5) modulation of CARD-4 mRNA or protein expression.

Stimulation of CARD-4 activity is desirable in situations in which CARD-4 is abnormally downregulated and/or in which increased CARD-4 activity is likely to have a beneficial effect. Conversely, inhibition of CARD-4 activity is desirable in situations in which CARD-4 is abnormally upregulated. e.g., in myocardial infarction, and/or in which decreased CARD-4 activity is likely to have a beneficial effect. Since CARD-4 may play be involved in the processing of cytokines, inhibiting the activity or expression of CARD-4 may be beneficial in patients that have aberrant inflammation.

The contents of all references, patents and published patent applications cited throughout this application are hereby incorporated by reference.

1. A method for identifying a candidate compound that modulates lipopolysaccharide (LPS)-mediated activation of NF-kB, the method comprising:
providing a cell that harbors LPS and expresses a polypeptide comprising a caspase recruitment domain (CARD), nucleotide binding site (NBS), or leucine rich repeat (LPR) domain of CARD-4;
exposing the cell to a test compound; and
measuring NF-kB activation in the cell;
wherein altered NF-kB activation in the presence of the test compound compared to NF-kB activation in the absence of the test compound indicates that the test compound is a candidate compound that modulates LPS-mediated activation of NF-kB.

2. The method of claim 1, wherein the cell is infected with Shigella flexneri.

3. The method of claim 1, wherein the cell is infected with Salmonella typhimurium.

4. The method of claim 1, wherein the cell is infected with Helicobacter pylori.

5. A method for identifying a candidate compound that modulates LPS-mediated activation of JNK kinase activity, the method comprising:
providing a cell that harbors LPS and expresses a polypeptide comprising a CARD, NBS, or LRR domain of CARD-4;
exposing the cell to a test compound; and
measuring JNK kinase activity in the cell;
wherein altered JNK kinase activity in the presence of the test compound compared to JNK kinase activity in the absence of the test compound indicates that the test compound is a candidate compound that modulates LPS-mediated activation of JNK kinase activity.

6. The method of claim 5, wherein the cell is infected with Shigella flexneri.

7. The method of claim 5, wherein the cell is infected with Salmonella typhimurium.

8. The method of claim 5, wherein the cell is infected with Helicobacter pylori.

9. A method for identifying a candidate compound that modulates an LPS-induced immune response, the method comprising:
providing a cell that expresses a polypeptide comprising a CARD, NBS, or LRR domain of CARD-4;
introducing LPS into the cell;
exposing the cell to a test compound; and
measuring oligomerization of the polypeptide in the cell;
wherein altered oligomerization of the polypeptide in the presence of the test compound compared to oligomerization of the polypeptide in the absence of the test compound indicates that the test compound is a candidate compound for modulating an LPS-induced immune response.

10. The method of claim 9, wherein the cell is infected with Shigella flexneri.

11. The method of claim 9, wherein the cell is infected with Salmonella typhimurium.

12. The method of claim 9, wherein the cell is infected with Helicobacter pylori.

13. A method of modulating LPS-induced activation of NF-kB or JNK, the method comprising:
providing a cell that harbors intracellular LPS; and
contacting the cell with a compound that modulates expression or activites of CARD-4 in an amount sufficient to modulate LPS-induced activation of NF-kB or JNK in the cell.

14. A method of modulating an LPS-induced immune response in an individual, the method comprising:
selecting an individual comprising cells harboring intracellular LPS; and
administering to the individual a compound that modulates expression or activity of CARD-4 in an amount sufficient to modulate an LPS-induced immune response in the individual.

15. The method of claim 14, wherein the individual is diagnosed as having a bacterial infection.

16. The method of claim 15, wherein the bacterial infection is a Shigella flexneri infection.

17. The method of claim 15, wherein the bacterial infection is a Salmonella typhimurium infection.

18. The method of claim 15, wherein the bacterial infection is a Helicobacter pylori infection.

19. A method of treating or preventing a bacterial infection, the method comprising:
selecting an individual having or at risk of having a bacterial infection;
administering to the individual a compound that modulates expression or activity of CARD-4 in an amount sufficient to treat or prevent the bacterial infection.

20. A mouse whose genome comprises a disruption in an endogenous CARD-4 gene, wherein said disruption results in decreased expression or a lack of expression of said endogenous CARD-4 gene, thereby causing a decreased ability of the mouse to clear a Salmonella typhimurium or Helicobacter pylori infection.
